(19) United States
(12) Patent Application Publication
(10) Pub. No.: US 2026/0132386 A1
(43) Pub. Date: May 14, 2026

(54) DEHYDROGENASE MUTANTS AND APPLICATIONS THEREOF IN AMINO ACID SYNTHESIS

(71) Applicant: METABOLIC EXPLORER, SAINT BEAUZIRE (FR)

(72) Inventors: Perrine VASSEUR, SAINT-LAURE (FR); Jean-Baptiste BOUDON, VOLVIC (FR); Laurence DUMON-SEIGNOVERT, PONT DU CHATEAU (FR); Céline RAYNAUD, SAINT BEAUZIRE (FR); Thomas DESFOUGERES, DISSAY (FR)

(73) Assignee: METABOLIC EXPLORER, SAINT BEAUZIRE (FR)

(21) Appl. No.: 18/686,216

(22) PCT Filed: Aug. 18, 2022
(86) PCT No.: PCT/EP2022/073084

§ 371 Date: Feb. 23, 2024

(30) Foreign Application Priority Data
Aug. 25, 2021 (EP) 21306148.4

(51) Int. Cl. C12N 9/06; C12N 1/20; C12P 13/06; C12R 1/19

(52) U.S. Cl. CPC C12N 9/0016; C12N 1/20; C12P 13/06; C12R 2001/19; C12Y 104/01008; C12Y 104/01009

(57) ABSTRACT

The present invention relates to a modified dehydrogenase, wherein one or more amino acid residues of the dehydrogenase are modified as compared to an unmodified dehydrogenase, and wherein the modified dehydrogenase has an increased ratio of leucine dehydrogenase or isoleucine dehydrogenase activity to valine dehydrogenase activity as compared to the unmodified dehydrogenase, and applications thereof.

### FIELD OF THE INVENTION

The present invention relates to dehydrogenase mutants and applications thereof in branched-chain amino acid synthesis.

### BACKGROUND OF THE INVENTION

Amino acids are used in many industrial fields, including the food, animal feed, cosmetics, pharmaceutical, and chemical industries and have an annual worldwide market growth rate of an estimated 5 to 7% (Leuchtenberger, et al., 2005). Among these, the leucine and isoleucine branched chain amino acids are particularly important for the nutrition of humans and a number of livestock species as they are essential amino acids that cannot be synthesized in mammals. As such, they are commonly used as food additives and in dietary supplements, with leucine also being used as a flavor enhancer. Branched chain amino acids also function as precursors in the synthesis of herbicides and antibiotics, such as polyketides.

Branched chain amino acids may be produced via chemical synthesis, extraction from protein hydrolysates, or microbial fermentation. Of these techniques, fermentation is the most commonly used today, due to the associated economic and environmental advantages. In particular, fermentation provides a useful way of using abundant, renewable, and/or inexpensive materials as the main source of carbon. Furthermore, while both D- and L-enantiomers are generated in equimolar amounts when using chemical synthesis, requiring additional downstream isolation of the L-enantiomer, fermentation produces only the L-enantiomer. Biosynthesis of leucine and isoleucine by fermentation is generally performed using microorganisms of the Corynebacterium or Escherichia genera, such as Corynebacterium glutamicum or Escherichia coli.

Originally, leucine and isoleucine producing strains were isolated by random mutagenesis. However, more recently, microorganisms have been subject to rational metabolic engineering, with strategies to improve amino acid production focusing mainly on removing feedback inhibition, modifying upstream central carbon flux, and reducing downstream synthesis of undesired by-products (see e.g., Yamamato et al., 2017, Park et al., 2010).

As an example, amino acid production may be improved by incorporation of feedback-resistant threonine dehydratase and aspartate kinase III (encoded by ilvA and lysC, respectively, in E. coli) for isoleucine, while removal of feedback inhibition of leuA may improve leucine production. As a further example, production may be improved by overexpressing the leuE gene encoding an L-leucine specific exporter or deleting the livK gene encoding an L-leucine specific transporter in E. coli (Park et al., 2010).

In view of the ever-increasing demand for leucine and isoleucine in industrial applications, there remains a need for further improvements in the production/isolation of these amino acids. In particular, there remains a need for modified enzymes and microorganisms allowing for improved production of leucine or isoleucine, as well as cost-effective, simple, and rapid methods for producing leucine or isoleucine on an industrial scale.

### BRIEF DESCRIPTION OF THE INVENTION

The present invention addresses the above needs, providing modified dehydrogenase enzymes having an increased ratio of leucine dehydrogenase or isoleucine dehydrogenase activity to valine dehydrogenase activity, as well as microorganisms incorporating said enzymes and corresponding methods for the production of leucine or isoleucine.

Specifically, a modified dehydrogenase is provided herein, wherein one or more amino acid residues of the dehydrogenase are modified as compared to an unmodified dehydrogenase, and wherein the modified dehydrogenase has an increased ratio of leucine dehydrogenase or isoleucine dehydrogenase activity to valine dehydrogenase activity as compared to the unmodified dehydrogenase.

Dehydrogenase enzymes having such modified activity are described for the first time here. The inventors have surprisingly found that these dehydrogenase enzymes advantageously provide similar or even increased yields of leucine or isoleucine amino acids, along with an increased titer of leucine or isoleucine as compared to valine, thereby facilitating downstream purification of these amino acids.

Preferably, the dehydrogenase is a leucine or valine dehydrogenase.

In one aspect, the dehydrogenase preferably has at least 95% sequence similarity to the dehydrogenase having the sequence of SEQ ID NO: 2 and comprises an alanine at the position corresponding to position 319, a threonine at the position corresponding to position 323, and/or a histidine at the position corresponding to position 330 of SEQ ID NO: 2.

Preferably, the dehydrogenase is selected from:
Vdh* (V319A) of sequence SEQ ID NO: 7,
Vdh* (I323T) of sequence SEQ ID NO: 9,
Vdh* (R330H) of sequence SEQ ID NO: 11,
Vdh* (V319A/I323T) of sequence SEQ ID NO: 13,
Vdh* (V319A/R330H) of sequence SEQ ID NO: 15,
Vdh* (I323T/R330H) of sequence SEQ ID NO: 17, and
Vdh* (V319A/I323T/R330H) of sequence SEQ ID NO: 19.

In another aspect, the dehydrogenase preferably has at least 95% sequence similarity to the dehydrogenase having the sequence of SEQ ID NO: 21 and comprises an alanine at the position corresponding to position 294, a threonine at the position corresponding to position 298, and/or a histidine at the position corresponding to position 305 of SEQ ID NO: 21.

Preferably, the dehydrogenase is selected from:
Ldh* (V294A) of sequence SEQ ID NO: 24,
Ldh* (L298T) of sequence SEQ ID NO: 26,
Ldh* (R305H) of sequence SEQ ID NO: 28,
Ldh* (V294A/L298T) of sequence SEQ ID NO: 30,
Ldh* (V294A/R305H) of sequence SEQ ID NO: 32,
Ldh* (L298T/R305H) of sequence SEQ ID NO: 34, and
Ldh* (V294A/L298T/R305H) of sequence SEQ ID NO: 36.

Preferably, the dehydrogenase is coded by a vdh* gene having the sequence of one of SEQ ID NOs: 6, 8, 10, 12, 14, 16, 18 or an ldh* gene having the sequence of one of SEQ ID NOs: 23, 25, 27, 29, 31, 33, 35.

The invention further relates to an Escherichia coli microorganism genetically modified for the increased production of a branched chain amino acid selected from leucine and isoleucine, wherein the microorganism further comprises the expression of at least one heterologous gene coding a modified dehydrogenase provided herein.

Preferably, the microorganism for the production of leucine comprises a deletion of at least one of the following genes: adhE, ldhA, and mgsA, and an overexpression of at least one of the following genes: ilvC, ilvD, ilvBN*, and leuA*BCD, wherein the ilvN* gene codes for a polypeptide having the sequence of SEQ ID NO: 50 and the leuA* gene codes for a polypeptide having the sequence of SEQ ID NO: 46.

Preferably, the microorganism for the production of isoleucine comprises a deletion of at least one of the following genes: lysA, metA, tdh and icIR genes and an overexpression of at least one of the following genes: ilvDA*, ilvC, ilvH*, Irp, thrA*BC, lysC*, ppc and ygaZH, wherein the ilvA* gene codes for a polypeptide having the sequence of SEQ ID NO: 70, wherein the lysC* gene codes for a polypeptide having the sequence of SEQ ID NO: 66, and wherein the ilvH* gene codes for a polypeptide having the sequence of SEQ ID NO: 90.

Preferably, the microorganism provided herein has been genetically modified to be able to utilize sucrose as a carbon source, preferably wherein said microorganism further comprises the overexpression of:
the heterologous cscBKAR genes of E. coli EC3132, or
the heterologous scrKYABR genes of Salmonella sp.

The invention further relates to a method for the fermentative production of a branched chain amino acid selected from leucine and isoleucine, comprising the steps of:
a) culturing, under fermentative conditions, an Escherichia coli microorganism as provided herein, in a culture medium comprising a carbohydrate as a source of carbon; and
b) recovering the branched chain amino acid from the culture medium.

Preferably, the source of carbon is glucose and/or sucrose.

Preferably, the method further comprises a step c) of purifying the branched chain amino acid recovered in step b).

### DETAILED DESCRIPTION

Before describing the present invention in detail, it is to be understood that the invention is not limited to particularly exemplified microorganism and/or methods and may, of course, vary. Indeed, various modifications, substitutions, omissions, and changes may be made without departing from the scope of the invention. It shall also be understood that the terminology used herein is for the purpose of describing particular embodiments of the invention only, and is not intended to be limiting.

All publications, patents and patent applications cited herein, whether supra or infra, are hereby incorporated by reference in their entirety. Furthermore, the practice of the present invention employs, unless otherwise indicated, conventional microbiological and molecular biological techniques that are within the skill of the art. Such techniques are well-known to the skilled person, and are fully explained in the literature.

Unless defined otherwise, all technical and scientific terms used herein have the same meanings as are commonly understood by one of ordinary skill in the art to which this invention belongs. Although any materials and methods similar or equivalent to those described herein can be used to practice or test the present invention, preferred material and methods are provided.

It must be noted that as used herein and in the appended claims, the singular forms “a,” “an,” and “the,” include plural reference unless the context clearly dictates otherwise. Thus, for example, a reference to “a microorganism” includes a plurality of such microorganisms, and so forth.

The terms “comprise,” “contain,” “include,” and variations thereof such as “comprising” are used herein in an inclusive sense, i.e., to specify the presence of the stated features but not to preclude the presence or addition of further features in various embodiments of the invention.

A first aspect of the invention concerns a modified dehydrogenase enzyme wherein one or more amino acid residues of the dehydrogenase are modified as compared to an unmodified dehydrogenase, and wherein the modified dehydrogenase has an increased ratio of leucine dehydrogenase or isoleucine dehydrogenase activity to valine dehydrogenase activity as compared to the unmodified dehydrogenase.

A “modified,” “mutated,” or “mutant” dehydrogenase refers to a dehydrogenase in which a mutation has been introduced into the corresponding protein sequence. The modified dehydrogenase of the present invention is a dehydrogenase enzyme comprising at least one amino acid difference, preferably at least two amino acid differences, more preferably at least three amino acid differences, when compared to the unmodified enzyme. More preferably, the mutant dehydrogenase comprises one, two, or three amino acid differences, when compared to the unmodified enzyme. Preferably, the amino acid difference is an amino acid substitution (i.e., the amino acid residue in the protein sequence is replaced by a different amino acid residue at the same position).

An “unmodified” enzyme (e.g., an unmodified dehydrogenase) refers to an enzyme prior to mutation, in other words, the “parent” enzyme. The unmodified enzyme may be of any origin. It may notably be natural or synthetic, and may more particularly correspond to a native or wild-type enzyme isolated from a microorganism, preferably a microorganism other than E. coli.

Identifying a modified dehydrogenase enzyme may be performed by screening native enzymes of various microorganisms for an increased ratio of leucine dehydrogenase or isoleucine dehydrogenase activity to valine dehydrogenase activity. Alternatively, mutation(s) may be induced in an enzyme of a given microorganism followed by selection of enzymes having an increased ratio of leucine dehydrogenase or isoleucine dehydrogenase activity to valine dehydrogenase activity. The skilled person is well-aware of methods that may be used to induce mutations. As a non-limiting example, mutations may be introduced by site-directed mutagenesis using, e.g., Polymerase Chain Reaction (PCR), by random mutagenesis techniques such as via mutagenic agents (Ultra-Violet rays or chemical agents like nitrosoguanidine (NTG) or ethylmethanesulfonate (EMS)), by DNA shuffling or error-prone PCR, or using culture conditions that apply a specific stress on the microorganism and induce mutagenesis.

In the present invention, the inventors have advantageously obtained several dehydrogenase mutants having an increased ratio of leucine dehydrogenase activity (LDH) or isoleucine dehydrogenase activity (lleDH) to valine dehydrogenase activity (VDH) (also expressed herein as the “LDH:VDH” and “lleDH:VDH” ratios, respectively). “Leucine dehydrogenase activity” or “LDH” as used herein refers to the activity of the dehydrogenase enzyme on ketoisocaproate (KIC) substrate to produce leucine. “Isoleucine dehydrogenase activity” or “lleDH” as used herein refers to the activity of the dehydrogenase enzyme on ketomethylvalerate (KMV) substrate to produce isoleucine. “Valine dehydrogenase activity” or “VDH” as used herein refers to the activity of the dehydrogenase enzyme on ketoisovalerate (KIV) substrate to produce valine. The LDH:VDH ratio thus refers to the ratio of enzymatic activity of the dehydrogenase on the KIC substrate relative to the KIV substrate. The lleDH:VDH ratio refers to the ratio of enzymatic activity of the dehydrogenase on the KMV substrate relative to the KIV substrate.

The dehydrogenase enzyme preferably belongs to KEGG enzyme class (EC) 1.4.1, more preferably EC 1.4.1.8, 1.4.1.9, or 1.4.1.23. Preferably, the modified dehydrogenase is a modified valine dehydrogenase (Vdh) or modified leucine dehydrogenase (Ldh), or a functional fragment or functional variant thereof. Preferably, the unmodified valine dehydrogenase is isolated from Streptomyces aureofaciens. Preferably, the unmodified leucine dehydrogenase is isolated from Thermoactinomyces intermedius.

A “functional fragment” of an enzyme (e.g., a dehydrogenase), as used herein, refers to parts of the amino acid sequence of the enzyme comprising at least all of the regions essential for exhibiting the biological activity of said enzyme. These parts of sequences can be of various lengths, provided that the biological activity of the amino acid sequence of the enzyme of reference is retained by said parts. In other words, a functional fragment of an enzyme as provided herein is enzymatically active.

A “functional variant” as used herein refers to a protein that is structurally different from the amino acid sequence of the enzyme (e.g., the dehydrogenase) but that generally retains all of the essential functional characteristics of said enzyme. A variant of a protein may be a naturally-occurring or a non-naturally occurring variant. Non-naturally occurring variants of a reference protein can be made, e.g., by mutagenesis techniques on the encoding nucleic acids or genes, e.g., by random mutagenesis or site-directed mutagenesis.

Structural differences may be limited in such a way that the amino acid sequence of the reference protein and the amino acid sequence of the variant may be highly similar overall, and identical in many regions. Structural differences may result from conservative or non-conservative amino acid substitutions, deletions and/or additions between the amino acid sequence of the reference protein and the variant. The only proviso is that, even if some amino acids are substituted, deleted and/or added, the biological activity of the amino acid sequence of the reference protein is retained by the variant. Such a variant of the dehydrogenase thus conserves the increased ratio of leucine dehydrogenase activity or isoleucine dehydrogenase activity to valine dehydrogenase activity. The activity ratio of the variants can be assessed according to in vitro tests known to the person skilled in the art.

Preferably, the modified valine dehydrogenase has at least 70%, 80%, 90%, 95%, 96%, or 97% sequence similarity or sequence identity to the dehydrogenase having the sequence of SEQ ID NO: 2. Preferably, the modified valine dehydrogenase comprises an alanine at the position corresponding to position 319, a threonine at the position corresponding to position 323, and/or a histidine at the position corresponding to position 330 of SEQ ID NO: 2.

Preferably, the dehydrogenase is selected from among:
Vdh* (V319A) of sequence SEQ ID NO: 7,
Vdh* (I323T) of SEQ ID NO: 9,
Vdh* (R330H) of sequence SEQ ID NO: 11,
Vdh* (V319A/I323T) of sequence SEQ ID NO: 13,
Vdh* (V319A/R330H) of sequence SEQ ID NO: 15,
Vdh* (I323T/R330H) of sequence SEQ ID NO: 17, and
Vdh* (V319A/I323T/R330H) of sequence SEQ ID NO: 19.

Preferably, the dehydrogenase is encoded by a vdh* gene having at least 70%, 80%, 90%, 95%, 96%, 97%, 98%, or 99% sequence identity with the sequence of SEQ ID NO: 1 or 3, wherein the vdh* gene codes for a protein having at least one of the substitutions V319A, I323T, and R330H with reference to the protein having the sequence of SEQ ID NO: 2. More preferably, the dehydrogenase is coded by a vdh* gene having the sequence of SEQ ID NO: 6, 8, 10, 12, 14, 16, or 18. Preferably, the unmodified (parent) dehydrogenase has the sequence of SEQ ID NO: 1 or 3.

Preferably, the modified leucine dehydrogenase has at least 70%, 80%, 90%, 95%, 96%, or 97% sequence similarity or sequence identity to the dehydrogenase having the sequence of SEQ ID NO: 21. Preferably the modified leucine dehydrogenase comprises an alanine at the position corresponding to position 294, a threonine at the position corresponding to position 298, and/or a histidine at the position corresponding to position 305 of SEQ ID NO: 21.

Preferably, the dehydrogenase is selected from among:
Ldh* (V294A) of sequence SEQ ID NO: 24,
Ldh* (L298T) of SEQ ID NO: 26,
Ldh* (R305H) of sequence SEQ ID NO: 28,
Ldh* (V294A/L298T) of sequence SEQ ID NO: 30,
Ldh* (V294A/R305H) of sequence SEQ ID NO: 32,
Ldh* (L298T/R305H) of sequence SEQ ID NO: 34, and
Ldh* (V294A/L298T/R305H) of sequence SEQ ID NO: 36.

Preferably, the dehydrogenase is encoded by an ldh* gene having at least 70%, 80%, 90%, 95%, 96%, 97%, 98%, or 99% sequence identity with the sequence of SEQ ID NO: 20 or 22, wherein the ldh* gene codes for a protein having at least one of the substitutions V294A, L298T, and R305H with reference to the protein having the sequence SEQ ID NO: 21. More preferably, the dehydrogenase is coded by an ldh* gene having the sequence of SEQ ID NO: 23, 25, 27, 29, 31, 33, or 35. Preferably, the unmodified (parent) dehydrogenase has the sequence of SEQ ID NO: 20 or 22.

The percentage of sequence identity between polypeptides or polynucleotides is a function of the number of identical amino acid residues or nucleotides at positions shared by the sequences of said polypeptides or polynucleotides. The term “sequence identity” or “identity” as used herein in the context of two nucleotide or amino acid sequences more particularly refers to the residues in the two sequences that are the identical when aligned for maximum correspondence. When percentage of sequence identity is used in reference to amino acid sequences, it is recognized that positions at which amino acids are not identical often differ by conservative amino acid substitutions, where amino acid residues are substituted for other amino acid residues having similar chemical properties (e.g., charge or hydrophobicity). Sequences that differ by such conservative substitutions are said to have “sequence similarity” or “similarity.” Thus, the degree of sequence similarity between polypeptides is a function of the number of similar amino acid residues at positions shared by the sequences of said proteins. Means of identifying similar sequences and their percent similarity or their percent identity are well-known to those skilled in the art, and include e.g., the BLAST programs, which can be used from the website http://www.ncbi.nlm.nih.gov/BLAST/with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given in GenBank for known genes, the person skilled in the art is able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well-known to those skilled in the art.

PFAM (protein family database of alignments and hidden Markov models; http://www.sanger.ac.uk/Software/Pfam/) represents a large collection of protein sequence alignments which may also be consulted by the skilled person. Each PFAM makes it possible to visualize multiple alignments, see protein domains, evaluate distribution among organisms, gain access to other databases, and visualize known protein structures.

Finally, COGs (clusters of orthologous groups of proteins; http://www.ncbi.nlm.nih.gov/COG/) may be obtained by comparing protein sequences from 43 fully sequenced genomes representing 30 major phylogenic lines. Each COG is defined from at least three lines, which permits the identification of former conserved domains.

Sequence similarity and sequence identity between amino acid or nucleotide sequences can be determined by comparing a position in each of the sequences which may be aligned for the purposes of comparison. When a position in the compared sequences is occupied by a similar amino acid or by the same amino acid then the sequences are, respectively, similar or identical at that position.

Percent similarity or percent identity as referred to herein are determined after optimal alignment of the sequences to be compared, which may therefore comprise one or more insertions, deletions, truncations and/or substitutions. Percent identity may be calculated by any sequence analysis method well-known to the person skilled in the art. The percent similarity or percent identity is preferably determined after global alignment of the sequences to be compared taken in their entirety over their entire length. In addition to manual comparison, it is possible to determine global alignment using the algorithm of Needleman and Wunsch (1970). Optimal alignment of sequences may preferably be conducted by the global alignment algorithm of Needleman and Wunsch (1970), by computerized implementations of this algorithm (such as CLUSTAL W) or by visual inspection.

For nucleotide sequences, sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4).

For amino acid sequences, sequence comparison may be performed using any software well-known to a person skilled in the art, such as the Needle software. The parameters used may notably be the following: “Gap open” equal to 10, “Gap extend” equal to 0.1, and the BLOSUM62 matrix.

As a particular example, to determine the percentage of similarity or identity between two amino acid sequences, the sequences are aligned for optimal comparison. For example, gaps can be introduced in the sequence of a first amino acid sequence for optimal alignment with the second amino acid sequence. The amino acid residues at corresponding amino acid positions are then compared. When a position in the first sequence is occupied by a different but conserved amino acid residue, the molecules are similar at that position, and accorded a particular score (e.g., as provided in a given amino acid substitution matrix, discussed below). When a position in the first sequence is occupied by the same amino acid residue as the corresponding position in the second sequence, the molecules are identical at that position.

The percentage of identity between the two sequences is a function of the number of identical positions shared by the sequences. Hence % identity=number of identical positions/total number of overlapping positions×100.

In other words, the percentage of sequence identity is calculated by comparing two optimally aligned sequences, determining the number of positions at which the identical amino acid occurs in both sequences to yield the number of matched positions, dividing the number of matched positions by the total number of positions and multiplying the result by 100 to yield the percentage of sequence identity.

Sequence similarity may be expressed as the percent similarity of a given amino acid sequence to that of another amino acid sequence. This refers to the similarity between sequences on the basis of a “similarity score” that is obtained using a particular amino acid substitution matrix. Such matrices and their use in quantifying the similarity between two sequences are well-known in the art and described, for example in Dayhoff et al., 1978, and in Henikoff and Henikoff, 1992. Preferably, the similarity score is determined using the BLOSUM62 matrix. Sequence similarity may be calculated from the alignment of two sequences, and is based on both a substitution score matrix and a gap penalty function.

As a non-limiting example, the similarity score is determined using the BLOSUM62 matrix, a gap existence penalty of 10, and a gap extension penalty of 0.1 or the BLOSUM62 matrix, a gap existence penalty of 11, and a gap extension penalty of 1. Preferably, no compositional adjustments are made to compensate for the amino acid compositions of the sequences being compared and no filters or masks (e.g., to mask off segments of the sequence having low compositional complexity) are applied when determining sequence similarity using web-based programs, such as BLAST. The maximum similarity score obtainable for a given amino acid sequence is that obtained when comparing the sequence with itself. For example, the maximum similarity score obtainable for SEQ ID NO: 2 is 693 and the maximum similarity score obtainable for SEQ ID NO: 21 is 669 using the above-described parameters (i.e., using the BLOSUM62 matrix, a gap existence penalty of 10, a gap extension penalty of 1, no compositional adjustment, and no filter or mask). The skilled person is able to determine such maximum similarity scores on the basis of the above-described parameters for any amino acid sequence. A statistically relevant similarity can furthermore be indicated by a “bit score” as described, for example, in Durbin et al., Biological Sequence Analysis, Cambridge University Press (1998).

To determine if a given amino acid sequence has at least e.g., 95% similarity with a protein provided herein, said amino acid sequence can be optimally aligned as provided above, preferably using the BLOSUM62 matrix, a gap existence penalty of 10, and a gap extension penalty of 1. Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. A sequence having 95% similarity with SEQ ID NO: 2 using the above-described parameters will have a score of at least 658. Vdh* (V319A/I323T/R330H) of sequence SEQ ID NO: 19 notably has a score of 689 and greater than 99% sequence identity with SEQ ID NO: 2. A sequence having 95% similarity with SEQ ID NO: 21 using the above-described parameters will have a score of at least 635. Ldh* (V294A/L298T/R305H) of sequence SEQ ID NO: 36 notably has a score of 664 and greater than 99% sequence identity with SEQ ID NO: 21. The skilled person is able to determine 95% similarity with a maximum score determined on the basis of the above-described parameters for any amino acid sequence.

A further aspect of the invention concerns a microorganism genetically modified for the increased production of a branched chain amino acid selected from leucine and isoleucine, wherein the microorganism further comprises the expression of at least one heterologous gene coding a modified dehydrogenase as provided herein.

The term “microorganism,” as used herein, refers to a living microscopic organism, which may be a single cell or a multicellular organism and which can generally be found in nature. The microorganism provided herein is preferably a bacterium, yeast, or fungus. Preferably, the microorganism is selected within the Enterobacteriaceae, Thermoactinomycetaceae, Bacillaceae, Streptomycetaceae, or Corynebacteriaceae family or from among yeast, preferably from within the Saccharomycesaceae family. More preferably, the microorganism is a species of Escherichia, Klebsiella, Bacillus, Thermoactinomyces, Streptomyces, Corynebacterium, or Saccharomyces. Even more preferably, said Enterobacteriaceae bacterium is Escherichia coli or Klebsiella pneumoniae, said Thermoactinomycetaceae bacterium is Thermoactinomyces intermedius, said Streptomycetaceae bacterium is Streptomyces aureofaciens, said Corynebacteriaceae bacterium is Corynebacterium glutamicum, or said Saccharomycesceae yeast is Saccharomyces cerevisiae. Most preferably, the microorganism of the invention is Escherichia coli.

The terms “recombinant microorganism,” “genetically modified microorganism,” or “microorganism genetically modified” are used interchangeably herein and refer to a microorganism or a strain of microorganism that has been genetically modified or genetically engineered. This means, according to the usual meaning of these terms, that the microorganism of the invention is not found in nature and is genetically modified when compared to the “parental” microorganism from which it is derived. The “parental” microorganism may occur in nature (i.e., a wild-type microorganism) or may have been previously modified. The recombinant microorganism of the invention may notably be modified by the introduction, deletion, and/or modification of genetic elements. Such modifications can be performed, e.g., by genetic engineering or by adaptation, wherein a microorganism is cultured in conditions that apply a specific stress on the microorganism and induce mutagenesis.

A microorganism genetically modified for the increased production of a branched chain amino acid selected from leucine and isoleucine means that said microorganism is a recombinant microorganism that has increased production of leucine or isoleucine as compared to a parent microorganism. In other words, said microorganism has been genetically modified for increased production of leucine or isoleucine.

The microorganism provided herein may notably comprise a modification such that the expression level of an endogenous gene is modulated. The term “endogenous gene” means that the gene was present in the microorganism before any genetic modification. Endogenous genes may be overexpressed by introducing heterologous sequences in addition to, or to replace, endogenous regulatory elements. Endogenous genes may also be overexpressed by introducing one or more supplementary copies of the gene into the chromosome or on a plasmid. In this case, the endogenous gene initially present in the microorganism may be deleted. Endogenous gene expression levels, protein expression levels, or the activity of the encoded protein, can also be increased or attenuated by introducing mutations into the coding sequence of a gene or into non-coding sequences. These mutations may be synonymous, when no modification in the corresponding amino acid occurs, or non-synonymous, when the corresponding amino acid is altered. Synonymous mutations do not have any impact on the function of translated proteins, but may have an impact on the regulation of the corresponding genes or even of other genes, if the mutated sequence is located in a binding site for a regulator factor. Non-synonymous mutations may have an impact on the function or activity of the translated protein as well as on regulation depending the nature of the mutated sequence.

In particular, mutations in non-coding sequences may be located upstream of the coding sequence (i.e., in the promoter region, in an enhancer, silencer, or insulator region, in a specific transcription factor binding site) or downstream of the coding sequence. Mutations introduced in the promoter region may be in the core promoter, proximal promoter or distal promoter. Mutations may be introduced by any of the methods described herein. The insertion of one or more supplementary nucleotide(s) in the region located upstream of a gene can notably modulate gene expression.

A particular way of modulating endogenous gene expression is to exchange the endogenous promoter of a gene (e.g., wild-type promoter) with a stronger or weaker promoter to upregulate or downregulate expression of the endogenous gene. The promoter may be endogenous (i.e., originating from the same species) or exogenous (i.e., originating from a different species). It is well within the ability of the person skilled in the art to select an appropriate promoter for modulating the expression of an endogenous gene. Such a promoter be, for example, a Ptrc, Ptac, Ptet, or Plac promoter, or a lambda P_{L} (PL) or lambda P_{R} (PL) promoter. The promoter may be “inducible” by a particular compound or by specific external conditions, such as temperature or light or a small molecule, such as an antibiotic.

A microorganism may also be genetically modified to express one or more exogenous or heterologous genes so as to overexpress the corresponding gene product (e.g., an enzyme). The terms “exogenous gene” or “heterologous gene” are used interchangeably herein and indicate that a gene was introduced into a microorganism wherein said gene is not naturally occurring in said microorganism. The gene coding for the modified dehydrogenase is a heterologous gene (i.e., the unmodified gene was isolated from T. intermedius or S. aureofaciens and subjected to codon-optimization and mutation prior to being introduced into E. coli). In particular, the exogenous gene may be directly integrated into the chromosome of the microorganism, or be expressed extra-chromosomally within the microorganism by plasmids or vectors. For successful expression, the exogenous gene(s) must be introduced into the microorganism with all of the regulatory elements necessary for their expression or be introduced into a microorganism that already comprises all of the regulatory elements necessary for their expression. The genetic modification or transformation of microorganisms with one or more exogenous genes is a routine task for those skilled in the art.

One or more copies of a given exogenous gene can be introduced on a chromosome by methods well-known in the art, such as by genetic recombination. When a gene is expressed extra-chromosomally, it can be carried by a plasmid or a vector. Different types of plasmid are notably available, which may differ in respect to their origin of replication and/or on their copy number in the cell. For example, a microorganism transformed by a plasmid can contain 1 to 5 copies of the plasmid, about 20 copies, or even up to 500 copies, depending on the nature of the selected plasmid. A variety of plasmids having different origins of replication and/or copy numbers are well-known in the art and can be easily selected by the skilled practitioner for such purposes, including, for example, pTrc, pACYC184, pBR322, pUC18, pUC19, pKC30, pRep4, pHS1, pHS2, or pPLc236.

It should be understood that, in the context of the present invention, when an exogenous gene encoding a protein of interest is expressed in a microorganism, such as E. coli, a synthetic version of this gene is preferably constructed by replacing non-preferred codons or less preferred codons with preferred codons of said microorganism which encode the same amino acid. Indeed, it is well-known in the art that codon usage varies between microorganism species, and that this may impact the recombinant expression level of a protein of interest. To overcome this issue, codon optimization methods have been developed, and are extensively described by Graf et al. (2000), Deml et al. (2001) and Davis & Olsen (2011). Several software programs have notably been developed for codon optimization determination such as the GeneOptimizer® software (Lifetechnologies) or the OptimumGene™ software of (GenScript). In other words, the exogenous gene encoding a protein of interest is preferably codon-optimized for expression in the chosen microorganism.

On the basis of a given amino acid sequence, the skilled person is furthermore able to identify an appropriate polynucleotide coding for said polypeptide (e.g., in the available databases, such as Uniprot), or to synthesize the corresponding polypeptide or a polynucleotide coding for said polypeptide. De novo synthesis of a polynucleotide can be performed, for example, by initially synthesizing individual nucleic acid oligonucleotides and hybridizing these with oligonucleotides complementary thereto, such that they form a double-stranded DNA molecule, and then ligating the individual double-stranded oligonucleotides such that the desired nucleic acid sequence is obtained.

The terms “expressing,” “overexpressing,” or “overexpression” of a protein of interest, such as an enzyme, refer herein to an increase in the expression level and/or activity of said protein in a microorganism, as compared to the corresponding parent microorganism that does not comprise the modification present in the genetically modified microorganism. A heterologous gene/protein can be considered to be “expressed” or “overexpressed” in a genetically modified microorganism when compared with a corresponding parent microorganism in which said heterologous gene/protein is absent. In contrast, the terms “attenuating” or “attenuation” of a protein of interest refer to a decrease in the expression level and/or activity of said protein in a microorganism, as compared to the parent microorganism. The attenuation of expression can notably be due to either the exchange of the wild-type promoter for a weaker natural or synthetic promoter or the use of an agent reducing gene expression, such as antisense RNA or interfering RNA (RNAi), and more particularly small interfering RNAs (siRNAs) or short hairpin RNAs (shRNAs). Promoter exchange may notably be achieved by the technique of homologous recombination (Datsenko & Wanner, 2000). The complete attenuation of the expression level and/or activity of a protein of interest means that expression and/or activity is abolished; thus, the expression level of said protein is null. The complete attenuation of the expression level and/or activity of a protein of interest may be due to the complete suppression of the expression of a gene. This suppression can be either an inhibition of the expression of the gene, a deletion of all or part of the promoter region necessary for expression of the gene, or a deletion of all or part of the coding region of the gene. A deleted gene can notably be replaced by a selection marker gene that facilitates the identification, isolation and purification of the modified microorganism. As a non-limiting example, suppression of gene expression may be achieved by the technique of homologous recombination, which is well-known to the person skilled in the art (Datsenko & Wanner, 2000).

Modulating the expression level of one or more proteins may thus occur by altering the expression of one or more endogenous genes that encode said protein within the microorganism as described above or by introducing one or more heterologous genes that encode said protein into the microorganism.

The term “expression level” as used herein, refers to the amount (e.g., relative amount, concentration) of a protein of interest (or of the gene encoding said protein) expressed in a microorganism, which is measurable by methods well-known in the art. The level of gene expression can be measured by various known methods including Northern blotting, quantitative RT-PCR, and the like. Alternatively, the level of expression of the protein coded by said gene may be measured, for example by SDS-PAGE, HPLC, LC/MS and other quantitative proteomic techniques (Bantscheff et al., 2007), or, when antibodies against said protein are available, by Western Blot-Immunoblot (Burnette, 1981), Enzyme-linked immunosorbent assay (e.g., ELISA) (Engvall and Perlman, 1971), protein immunoprecipitation, immunoelectrophoresis, and the like. The copy number of an expressed gene can be quantified, for example, by restricting chromosomal DNA followed by Southern blotting using a probe based on the gene sequence, fluorescence in situ hybridization (FISH), RT-qPCR, and the like.

Overexpression of a given gene or the corresponding protein may be verified by comparing the expression level of said gene or protein in the genetically modified organism to the expression level of the same gene or protein in a control microorganism that does not have the genetic modification (i.e., the parental strain).

The recombinant microorganism notably expresses at least one heterologous gene coding a modified dehydrogenase as provided herein. Indeed, the expression of modified dehydrogenase advantageously increases the titer of leucine or isoleucine produced by the microorganism relative to the titer of valine. The expression of modified dehydrogenase may furthermore advantageously increase leucine or isoleucine yield.

The microorganism for the production of leucine preferably comprises an attenuation of the expression of one or more of the following proteins: lactate dehydrogenase (LdhA), alcohol dehydrogenase (AdhE), methylglyoxal synthase (MgsA), fumarate reductase enzyme complex (FrdABCD), pyruvate formate lyase (PfIAB), and/or acetate kinase (AckA) and phosphate acetyltransferase (Pta). Said genes are notably endogenous in E. coli.

Preferably, LdhA has at least 80%, 90%, 95%, or 100% sequence similarity or sequence identity with the sequence of SEQ ID NO: 38. Preferably, AdhE has at least 80%, 90%, 95%, or 100% sequence similarity or sequence identity with the sequence of SEQ ID NO: 40. Preferably, MgsA has at least 80%, 90%, 95%, or 100% sequence similarity or sequence identity with the sequence of SEQ ID NO: 42. Preferably, FrdA, FrdB, FrdC, and FrdD have at least 80%, 90%, 95%, or 100% sequence similarity or sequence identity with the sequence of SEQ ID NO: 100, 102, 104 and 106, respectively. Preferably, PflA and PflB have at least 80%, 90%, 95%, or 100% sequence similarity or sequence identity with the sequence of SEQ ID NO: 108 and 110, respectively. Preferably, AckA has at least 80%, 90%, 95%, or 100% sequence similarity or sequence identity with the sequence of SEQ ID NO: 112. Preferably Pta has at least 80%, 90%, 95%, or 100% sequence similarity or sequence identity with the sequence of SEQ ID NO: 114.

Preferably, attenuation of expression results from a partial or complete deletion of the gene encoding said protein (i.e., ldhA, adhE, mgsA, frdABCD, pflAB, and/or ackA-pta genes). Preferably, the ldhA gene has at least 80%, 90%, 95%, or 100% sequence identity with the sequence of SEQ ID NO: 37. Preferably, the adhE gene has at least 80%, 90%, 95%, or 100% sequence identity with the sequence of SEQ ID NO: 39. Preferably, the mgsA gene has at least 80%, 90%, 95%, or 100% sequence identity with the sequence of SEQ ID NO: 41. Preferably, the frdABCD genes have at least 80%, 90%, 95%, or 100% sequence identity with the sequences of SEQ ID NOs: 99, 101, 103, and 105, respectively. Preferably, the pflAB genes have at least 80%, 90%, 95%, or 100% sequence identity with the sequences of SEQ ID NOs: 107 and 109, respectively. Preferably, the ackA-pta genes have at least 80%, 90%, 95%, or 100% sequence identity with the sequences of SEQ ID NOS: 111 and 113, respectively.

The microorganism for the production of leucine may further comprise an overexpression of one or more of the following proteins: ketol-acid reductoisomerase (NADP(+)) (llvC), dihydroxy-acid dehydratase (llvD), acetolactate synthase (llvBN*), 2-isopropylmalate synthase (LeuA*), 3-isopropylmalate dehydrogenase (LeuB), 3-isopropylmalate dehydratase (LeuCD), and leucine efflux protein (LeuE), wherein llvN* and LeuA* are feedback resistant proteins.

The term “feedback resistant protein” as used herein refers to a protein which has been modified such that feedback inhibition of the protein (i.e., the reduction in enzyme activity mediated by the binding of the product to the enzyme) is reduced or even eliminated.

Preferably, llvC has at least 80%, 90%, 95%, or 100% sequence similarity or sequence identity with the sequence of SEQ ID NO: 72. Preferably, llvD has at least 80%, 90%, 95%, or 100% sequence similarity or sequence identity with the sequence of SEQ ID NO: 78. Preferably, IlvB and llvN* have at least 80%, 90%, 95%, or 100% sequence similarity or sequence identity with the sequence of SEQ ID NOs: 80 and 50, respectively, with IlvN* comprising the substitutions G20D, V21D and M22F in cases where the sequence is not 100% identical to SEQ ID NO: 50. Preferably, LeuA*, LeuB, LeuC, and LeuD have at least 80%, 90%, 95%, or 100% sequence similarity or sequence identity with the sequence of SEQ ID NO: 46, 82, 84, and 86, respectively, with LeuA comprising the substitution G479C in cases where the sequence is not 100% identical to SEQ ID NO: 46. Preferably, LeuE has at least 80%, 90%, 95%, or 100% sequence similarity or sequence identity with the sequence of SEQ ID NO: 116.

Preferably, the overexpression of said one or more proteins results from an overexpression of the gene coding said protein (i.e., ilvC, ilvD, ilvBN*, leuA*BCD, and/or leuE genes). Preferably, the ilvC gene has at least 80%, 90%, 95%, or 100% sequence identity with the sequence of SEQ ID NO: 71. Preferably, the ilvD gene has at least 80%, 90%, 95%, or 100% sequence identity with the sequence of SEQ ID NO: 77. Preferably, the ilvB and ilvN* genes have at least 80%, 90%, 95%, or 100% sequence identity with the sequences of SEQ ID NOs: 79 and 49, respectively, wherein the ilvN* gene codes for an amino acid having the substitutions G20D, V21D and M22F with reference to the wild-type IlvN protein having the sequence SEQ ID NO: 48; said wild-type llvN protein being coded by the ilvN gene of sequence SEQ ID NO: 47. Preferably, the leuA*BCD genes have at least 80%, 90%, 95%, or 100% sequence identity with the sequences of SEQ ID NOs: 45, 81, 83, and 85, respectively, wherein the leuA* gene codes for an amino acid having the substitution G479C with reference to the wild-type LeuA protein having the sequence SEQ ID NO: 44; said wild-type LeuA protein being coded by the leuA gene of sequence SEQ ID NO: 43. Preferably, the leuE gene has at least 80%, 90%, 95%, or 100% sequence identity with the sequence of SEQ ID NO: 115. Preferably, overexpression of an endogenous gene occurs by replacing the native promoter with an artificial promoter, such as the Ptrc promoter.

The microorganism for the production of isoleucine preferably comprises an attenuation in the expression of one or more of the following proteins: diaminopimelate decarboxylase (LysA), the homoserine O-succinyltransferase (MetA), the threonine dehydrogenase (Tdh), the “Isocitrate lyase Regulator” transcriptional regulator (IcIR). Said genes are notably endogenous in E. coli.

Preferably, LysA has at least 80%, 90%, 95%, or 100% sequence similarity or sequence identity with the sequence of SEQ ID NO: 52. Preferably, MetA has at least 80%, 90%, 95%, or 100% sequence similarity or sequence identity with the sequence of SEQ ID NO: 54. Preferably, Tdh has at least 80%, 90%, 95%, or 100% sequence similarity or sequence identity with the sequence of SEQ ID NO: 56. Preferably, IcIR has at least 80%, 90%, 95%, or 100% sequence similarity or sequence identity with the sequence of SEQ ID NO: 58.

Preferably, attenuation of expression results from a partial or complete deletion of the gene encoding said protein (i.e., lysA, metA, tdh, and/or icIR genes). Preferably, the lysA gene has at least 80%, 90%, 95%, or 100% sequence identity with the sequence of SEQ ID NO: 51. Preferably, the metA gene has at least 80%, 90%, 95%, or 100% sequence identity with the sequence of SEQ ID NO: 53. Preferably, the tdh gene has at least 80%, 90%, 95%, or 100% sequence identity with the sequence of SEQ ID NO: 55. Preferably, the icIR gene has at least 80%, 90%, 95%, or 100% sequence identity with the sequence of SEQ ID NO: 57.

The microorganism for the production of isoleucine may further comprise an overexpression of one or more of the following proteins: llvD, threonine dehydratase (llvA*), llvC, acetolactate synthase (llvH*), leucine-responsive regulatory protein (Lrp), aspartokinase I (ThrA*), homoserine kinase (ThrB), threonine synthase (ThrC), aspartokinase 3 (LysC*), phosphoenolpyruvate carboxylase (Ppc), and L-valine exporter (YgaZH), wherein llvA*, llvH*, ThrA*, and LysC* are feedback resistant proteins.

Preferably, llvD has at least 80%, 90%, 95%, or 100% sequence similarity or sequence identity with the sequence of SEQ ID NO: 78. Preferably, llvA* has at least 80%, 90%, 95%, or 100% sequence similarity or sequence identity with the sequence of SEQ ID NO: 70, respectively, with llvA comprising the substitutions L447F, L451A in cases where the sequence is not 100% identical to SEQ ID NO: 70. Preferably, llvC has at least 80%, 90%, 95%, or 100% sequence similarity or sequence identity with the sequence of SEQ ID NO: 72. Preferably, llvH* has at least 80%, 90%, 95%, or 100% sequence similarity or sequence identity with the sequence of SEQ ID NO: 90, respectively, with llvH* comprising the substitutions G14D and S17F in cases where the sequence is not 100% identical to SEQ ID NO: 90. Preferably, Lrp has at least 80%, 90%, 95%, or 100% sequence similarity or sequence identity with the sequence of SEQ ID NO: 76. Preferably, ThrA* has at least 80%, 90%, 95%, or 100% sequence similarity or sequence identity with the sequence of SEQ ID NO: 62, respectively, with ThrA* comprising the substitution S345F in cases where the sequence is not 100% identical to SEQ ID NO: 62. Preferably, ThrB has at least 80%, 90%, 95%, or 100% sequence similarity or sequence identity with the sequence of SEQ ID NO: 92. Preferably, ThrC has at least 80%, 90%, 95%, or 100% sequence similarity or sequence identity with the sequence of SEQ ID NO: 94. Preferably, LysC* has at least 80%, 90%, 95%, or 100% sequence similarity or sequence identity with the sequence of SEQ ID NO: 66, respectively, with LysC* comprising the substitution T3521 in cases where the sequence is not 100% identical to SEQ ID NO: 66. Preferably, Ppc has at least 80%, 90%, 95%, or 100% sequence similarity or sequence identity with the sequence of SEQ ID NO: 74. Preferably, YgaZ and YgaH have at least 80%, 90%, 95%, or 100% sequence similarity or sequence identity with the sequences of SEQ ID NOs: 96 and 98 respectively.

Preferably, the overexpression of said one or more proteins results from an overexpression of the gene coding said protein (i.e., ilvDA*, ilvC, ilvH*, Irp, thrA*BC, lysC*, ppc, and/or ygaZH genes). Preferably, the ilvD gene has at least 80%, 90%, 95%, or 100% sequence identity with the sequence of SEQ ID NO: 77. Preferably, the ilvA* gene has at least 80%, 90%, 95%, or 100% sequence identity with the sequence of SEQ ID NO: 69, wherein the ilvA* gene codes for an amino acid having the substitutions L447F, L451A with reference to the wild-type llvA protein having the sequence SEQ ID NO: 68; said wild-type IlvA protein being coded by the ilvA gene of sequence SEQ ID NO: 67. Preferably, the ilvC gene has at least 80%, 90%, 95%, or 100% sequence identity with the sequence of SEQ ID NO: 71. Preferably, the ilvH* gene has at least 80%, 90%, 95%, or 100% sequence identity with the sequence of SEQ ID NO: 89, wherein the ilvH* gene codes for an amino acid having the substitution(s) G14D, S17F with reference to the wild-type llvH protein having the sequence SEQ ID NO: 88; said wild-type llvH protein being coded by the ilvH gene of sequence SEQ ID NO: 87. Preferably, the lrp gene has at least 80%, 90%, 95%, or 100% sequence identity with the sequence of SEQ ID NO: 75. Preferably, the thrA* gene has at least 80%, 90%, 95%, or 100% sequence identity with the sequence of SEQ ID NO: 61, wherein the thrA* gene codes for an amino acid having the substitution S345F with reference to the wild-type ThrA protein having the sequence SEQ ID NO: 60; said wild-type ThrA protein being coded by the thrA gene of sequence SEQ ID NO: 59. Preferably, the thrB gene has at least 80%, 90%, 95%, or 100% sequence identity with the sequence of SEQ ID NO: 91. Preferably, the thrC gene has at least 80%, 90%, 95%, or 100% sequence identity with the sequence of SEQ ID NO: 93. Preferably, the lysC* gene has at least 80%, 90%, 95%, or 100% sequence identity with the sequence of SEQ ID NO: 65, wherein the lysC* gene codes for an amino acid having the substitution T3521 with reference to the wild-type LysC protein having the sequence SEQ ID NO: 64; said wild-type LysC protein being coded by the lysC gene of sequence SEQ ID NO: 63. Preferably, the ppc gene has at least 80%, 90%, 95%, or 100% sequence identity with the sequence of SEQ ID NO: 73. Preferably, the ygaZH genes have at least 80%, 90%, 95%, or 100% sequence identity with the sequences of SEQ ID NOs: 95 and 97, respectively.

Preferably, overexpression of an endogenous gene occurs by replacing the native promoter with an artificial promoter, such as the Ptrc promoter.

Preferably, one or more of any of the above feedback-resistant proteins replaces the corresponding wild-type protein in the microorganism (e.g., llvN* replaces wild-type llvN in the microorganism). As a non-limiting example, the wild-type protein may be replaced with the feedback resistant mutant by deleting the gene coding for the wild-type protein in the microorganism and incorporating the gene coding for the feedback resistant mutant (e.g., by transforming the microorganism with a plasmid which overexpresses the gene) or by directly mutating the wild-type gene present in the microorganism such that it becomes feedback resistant.

In a further aspect, the microorganism genetically modified for the production of leucine or isoleucine as described herein is further modified to be able to use sucrose as a carbon source. Preferably, proteins involved in the import and metabolism of sucrose are overexpressed. Preferably, the following proteins are overexpressed:
CscB sucrose permease, CscA sucrose hydrolase, CscK fructokinase, and CscR csc-specific repressor, or
ScrA Enzyme II of the phosphoenolpyruvate-dependent phosphotransferase system and, said ScrK gene encodes ATP-dependent fructokinase, said ScrB sucrose 6-phosphate hydrolase (invertase), said ScrY sucrose porine, ScrR sucrose operon repressor.

Preferably, genes coding for said proteins are overexpressed according to one of the methods provided herein. Preferably, the microorganism overexpresses:
the heterologous cscBKAR genes of E. coli EC3132, or
the heterologous scrKYABR genes of Salmonella sp.

According to a further aspect, the present invention relates to a method for the fermentative production of a branched chain amino acid selected from leucine and isoleucine using the microorganism described herein. Said method comprises the steps of:
a) culturing, under fermentative conditions, the microorganism provided herein in a culture medium comprising a carbohydrate as a source of carbon; and
b) recovering the branched chain amino acid from the culture medium.

According to the invention, the terms “fermentative process,” “fermentative production,” “fermentation,” or “culture” are used interchangeably to denote the growth of microorganism. This growth is generally conducted in fermenters with an appropriate growth medium adapted to the microorganism being used.

An “appropriate culture medium” or a “culture medium” refers to a culture medium optimized for the growth of the microorganism and the synthesis of leucine or isoleucine by the cells. The culture medium (e.g., a sterile, liquid media) comprises nutrients essential or beneficial to the maintenance and/or growth of the microorganism such as carbon sources or carbon substrates, nitrogen sources; phosphorus sources, for example, monopotassium phosphate or dipotassium phosphate; trace elements (e.g., metal salts, for example magnesium salts, cobalt salts and/or manganese salts); as well as growth factors such as amino acids and vitamins. The fermentation process is generally conducted in reactors with a synthetic, particularly inorganic, culture medium of known defined composition adapted to the microorganism, e.g., E. coli. In particular, the inorganic culture medium can be of identical or similar composition to an M9 medium (Anderson, 1946), an M63 medium (Miller, 1992) or a medium such as defined by Schaefer et al. (1999). “Synthetic medium” refers to a culture medium comprising a chemically defined composition on which organisms are grown.

The term “source of carbon,” “carbon source,” or “carbon substrate” according to the present invention refers to any carbon source capable of being metabolized by a microorganism wherein the substrate contains at least one carbon atom. According to the present invention, said source of carbon is preferably at least one carbohydrate, and in some cases a mixture of at least two carbohydrates. In one aspect, said source of carbon is a combination of at least one carbohydrate, such as glucose, and acetate.

The term “carbohydrate” refers to any carbon source capable of being metabolized by a microorganism and containing at least three carbon atoms, two atoms of hydrogen. The one or more carbohydrates may be selected from among the group consisting of: monosaccharides such as glucose, fructose, mannose, xylose, arabinose, galactose and the like, disaccharides such as sucrose, cellobiose, maltose, lactose and the like, oligosaccharides such as raffinose, stacchyose, maltodextrins and the like, polysaccharides such as cellulose, hemicellulose, starch and the like, and glycerol. Especially preferred carbon sources are glycerol, arabinose, fructose, galactose, glucose, lactose, maltose, sucrose, xylose or a mixture of two or more thereof. Preferably, the carbohydrate is glycerol and/or glucose and/or sucrose, more preferably glucose and/or sucrose.

The term “source of nitrogen” according to the present invention refers to any nitrogen source capable of being used by the microorganism. Said source of nitrogen may be inorganic (e.g., (NH₄)₂SO₄) or organic (e.g., urea or glutamate). Preferably, said source of nitrogen is in the form of ammonium or ammoniac. Preferably, said source of nitrogen is either an ammonium salt, such as ammonium sulfate, ammonium chloride, ammonium nitrate, ammonium hydroxide and ammonium phosphate, or to ammoniac gas, corn steep liquor, peptone (e.g., Bacto™ peptone), yeast extract, meat extract, malt extract, or urea, or any combination thereof. In some cases, the nitrogen source may be derived from renewable biomass of microbial origin (such as beer yeast autolysate, waste yeast autolysate, baker's yeast, hydrolyzed waste cells, algae biomass), vegetal origin (such as cotton seed meal, soy peptone, soybean peptide, soy flour, soybean flour, soy molasses, rapeseed meal, peanut meal, wheat bran hydro lysate, rice bran and defatted rice bran, malt sprout, red lentil flour, black gram, bengal gram, green gram, bean flour, flour of pigeon pea, protamylasse) or animal origin (such as fish waste hydrolysate, fish protein hydrolysate, chicken feather; feather hydrolysate, meat and bone meal, silk worm larvae, silk fibroin powder, shrimp wastes, beef extract), or any other nitrogen containing waste. More preferably, said source of nitrogen is peptone and/or yeast extract.

According to a particularly preferred embodiment, the culture medium comprises at least one carbohydrate, such as glucose, as well as acetate and/or yeast extract and/or peptone.

The person skilled in the art is able to define the culture conditions for the microorganisms according to the invention. In particular the bacteria are fermented at a temperature between 20° C. and 55° C., preferably between 25° C. and 40° C., more preferably between about 30° C. to 39° C., even more preferably about 37° C. In cases, where a thermo-inducible promoter is comprised in the microorganism provided herein, said microorganism is preferably fermented at about 39° C.

This process can be carried out either in a batch process, in a fed-batch process or in a continuous process. It can be carried out under aerobic, micro-aerobic or anaerobic conditions, or a combination thereof (for example, aerobic conditions followed by anaerobic conditions).

“Under aerobic conditions” means that oxygen is provided to the culture by dissolving the gas into the liquid phase. This could be obtained by (1) sparging oxygen containing gas (e.g., air) into the liquid phase or (2) shaking the vessel containing the culture medium in order to transfer the oxygen contained in the head space into the liquid phase. The main advantage of the fermentation under aerobic conditions is that the presence of oxygen as an electron acceptor improves the capacity of the strain to produce more energy under the form of ATP for cellular processes. Therefore, the strain has its general metabolism improved.

Micro-aerobic conditions are defined as culture conditions wherein low percentages of oxygen (e.g., using a mixture of gas containing between 0.1 and 15% of oxygen, completed to 100% with inert gas such as nitrogen, helium or argon, etc.), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The term “recovering” as used herein designates the process of separating or isolating the produced leucine or isoleucine by using conventional laboratory techniques known to the person skilled in the art. Leucine or isoleucine may by recovered from the culture medium and/or from the microorganism itself. Preferably, leucine or isoleucine is recovered from at least the culture medium.

The method provided herein may further comprise a step c) of purifying the branched chain amino acid recovered in step b). Methods for recovering and eventually purifying a branched chain amino acid from a fermentation medium are known to the skilled person. Purification may notably be performed using conventional laboratory techniques such as filtration, ion exchange, crystallization, and/or distillation.

### EXAMPLES

The present invention is further defined in the following examples. It should be understood that these examples, while indicating preferred embodiments of the invention, are given by way of illustration only. The person skilled in the art will readily understand that these examples are not limitative and that various modifications, substitutions, omissions, and changes may be made without departing from the scope of the invention.

### Methods

The protocols used in the following examples are:
Protocol 1 (Chromosomal modifications by homologous recombination, selection of recombinants and antibiotic cassette excision flanked by FRT sequences) and protocol 2 (Transduction of phage P1) used in this invention have been fully described in patent application WO2013/001055 (see in particular the “Examples Protocols” section and Examples 1 to 8, incorporated herein by reference).
Protocol 3: Construction of recombinant plasmids.

Recombinant DNA technology is well described and known to the person skilled in the art. Briefly, DNA fragments were PCR amplified using oligonucleotides (that the person skilled in the art will be able to define) and E. coli MG1655 genomic DNA or an adequate synthetically synthesized fragment was used as a matrix. The DNA fragments and chosen plasmid were digested with compatible restriction enzymes (that the person skilled in the art is able to define), then ligated and transformed into competent cells. Transformants were analyzed and recombinant plasmids of interest were verified by DNA sequencing.
Protocol 4: Directed mutagenesis

Those of ordinary skill in the art can easily use known methods, such as point mutation methods using oligonucleotides carrying the desired mutation, to mutate the nucleotide sequence encoding the protein of interest.
Protocol 5: Random mutagenesis

Several protocols are described and known to the person skilled in the art. Briefly, mutant fragments were obtained by error-prone PCR amplification of a gene of interest (vdh) using the DiversifyR PCR Random Mutagenesis Kit from Clontech™. By adjusting the concentration of MnSO4, libraries with different mutation frequencies were obtained. The purified mutant fragments were cloned into a vector and transformed into E. coli DH5alpha commercial competent cells to obtain mutation libraries of the gene of interest.
Protocol 6: Determination of valine dehydrogenase (VDH), leucine dehydrogenase (LDH) and isoleucine dehydrogenase (lleDH) enzymatic activities

Single colonies were picked and cultured in 96 deep-well plates containing LB liquid medium (10 g/L bactopeptone, 5 g/L yeast extract, 5 g/L NaCl) supplemented with 5 g/L of glucose and adequate antibiotic, at 39° C. for 16 h. After growth, cells were collected by centrifugation and proteins were extracted using the BugBuster 10× kit from Novagen™ complemented with lysozyme and DNAse I. A constant volume of supernatant was used for the determination of amino acid dehydrogenase activity (valine or leucine or isoleucine dehydrogenase) in a 96-well microtiter plate according to method described by Ohashima & Soda, 1979, except that ketoisovalerate (KIV), ketoisocaproate (KIC), or ketomethylvalerate (KMV) (for valine, leucine, or isoleucine dehydrogenase, respectively) was used as substrate at a concentration of 1.5 mM (instead of pyruvate for alanine dehydrogenase).

The amino acid dehydrogenase activities were expressed in enzyme activity units also named International Units (IU) according the following definition: 1 enzyme activity unit is the amount of enzyme required to transform 1 μmol of substrate per min. The milli International Unit (mIU) is one-thousandth of the International Unit (IU).
For valine dehydrogenase activity (VDH): substrate is ketoisovalerate (KIV) and valine is produced.
For leucine dehydrogenase activity (LDH): substrate is ketoisocaproate (KIC) and leucine is produced.
For isoleucine dehydrogenase activity (lleDH): substrate is ketomethylvalerate (KMV) and isoleucine is produced.

For a same supernatant, the ratio between the LDH or lleDH and VDH activities (referred to herein as “R LDH:VDH” or “R lleDH:VDH”) is expressed as follows:

$$R LDH : VDH = \frac{LDH ( mIU )}{VDH ( mIU )}$$

$$R IleDH : VDH = \frac{IleDH ( mIU )}{VDH ( mIU )}$$

The same protocol was used to measure the VDH, LDH, and lleDH activities of mutants constructed by directed mutagenesis.
Protocol 7: Evaluation of L-leucine and L-isoleucine fermentation performance

Production strains were evaluated in 500 mL Erlenmeyer baffled flasks using medium MM_LEF4 (Table 1) for leucine fermentation or medium MM_ILF2 (Table 2) for isoleucine fermentation, both adjusted to pH 6.8. A 5 mL preculture was grown at 30° C. for 16 hours in a rich medium (LB medium with 5 g·L⁻¹ glucose). It was used to inoculate a 50 mL culture to an OD₆₀₀ of 0.2. When necessary, antibiotics were added to the medium (spectinomycin at a final concentration of 50 mg·L⁻¹). The temperature of the cultures was 39° C. When the culture reached an OD600 of 2 to 5 uOD (OD unit), extracellular amino acids were quantified by HPLC after OPA/Fmoc derivatization and other relevant metabolites were analysed using HPLC with refractometric detection (organic acids and glucose).

TABLE 1
Composition of MM_LEF4 medium.

| Compound | Concentration (g · L⁻¹) |
|---|---|
| Citric acid•H₂O | 1.00 |
| MgSO₄•7H₂O | 1.00 |
| CaCl₂•2H₂O | 0.04 |
| CoCl₂•6H₂O | 0.0080 |
| MnSO₄•H₂O | 0.0200 |
| CuCl₂•2H₂O | 0.0020 |
| H₃BO₃ | 0.0010 |
| Na₂MoO₄•2H₂O | 0.0004 |
| ZnSO₄•7H₂O | 0.0040 |
| Na₂HPO₄ | 2.00 |
| K₂HPO₄ | 2.00 |
| (NH₄)₂HPO₄ | 8.00 |
| (NH₄)₂SO₄ | 5.00 |
| NH₄Cl | 0.13 |
| FeSO₄•7H₂O | 0.01 |
| Thiamin | 0.0116 |
| MOPS | 40 |
| Glucose | 20 |

TABLE 2
Composition of MM_ILF2 medium.

| Compound | Concentration (g · L⁻¹) |
|---|---|
| Citric acid•H₂O | 1.00 |
| MgSO₄•7H₂O | 1.00 |
| CaCl₂•2H₂O | 0.04 |
| CoCl₂•6H₂O | 0.0080 |
| MnSO₄•H₂O | 0.0200 |
| CuCl₂•2H₂O | 0.0020 |
| H₃BO₃ | 0.0010 |
| Na₂MoO₄•2H₂O | 0.0004 |
| ZnSO₄•7H₂O | 0.0040 |
| Na₂HPO₄ | 2.00 |
| K₂HPO₄ | 2.00 |
| (NH₄)₂HPO₄ | 8.00 |
| (NH₄)₂SO₄ | 5.00 |
| (NH₄)₂SO₄ | 0.13 |
| NH₄Cl | 0.01 |
| Thiamin | 0.0116 |
| MOPS | 40 |
| Glucose | 20 |
| Lysine | 0.2 |
| Methionine | 0.2 |

In these cultures, the leucine yield (YLeu) was expressed as follows:

$$YLeu ( \frac{g}{g} ) = \frac{Leucine produced ( g )}{Glucose consummed ( g )} \times 100$$

and the isoleucine yield (Ylle) was expressed as follows:

$$YIle ( \frac{g}{g} ) = \frac{isoleucine produced ( g )}{Glucose consummed ( g )} \times 100$$

### Example 1: Construction and Screening of vdh Gene Mutation Library

The amino acid dehydrogenase encoded by the vdh gene from Streptomyces aureofaciens (SEQ ID NO: 2, Uniprot A0A1E7N318, coded by the vdh gene of SEQ ID NO: 1) is more active on ketoisovalerate (KIV) than on ketoisocaproate (KIC) or on ketomethylvalerate (KMV). In order to obtain a dehydrogenase with less difference between its activity on KIV and KIC or KMV, and even with a greater activity on KIC and KMV than on KIV, a vdh gene mutation library was generated, screened, and mutants with improved ratios of LDH:VDH and lleDH:VDH activities were selected.

### Construction of vdh Gene Mutation Library

The vdh gene was synthetically synthesized with the codon usage being optimized to E. coli (SEQ ID NO: 3). Using protocol 3, the optimized vdh gene having the sequence of SEQ ID NO: 3, coding for wildtype vdh protein, was cloned under the PR promoter together with the cl857 allele of the thermosensitive repressor of lambda phage having the sequence of SEQ ID NO: 4 (amplified from the pFC1 vector, Mermet-Bouvier & Chauvat, 1994) into the pCL1920 vector (Lerner & Inouye, 1990), giving the “plasmid 1” plasmid. Mutated vdh gene fragments were obtained according to protocol 5. Purified mutant vdh gene fragments were cloned into plasmid 1 (instead of the optimized vdh gene coding for the wildtype Vdh protein) according to protocol 3 and transformed into E. coli DH5alpha commercial competent cells to obtain vdh gene mutation libraries.

### Screening of the vdh Gene Mutation Library

Around 6000 clones were screened according to protocol 6. Of these, three mutants having a significant improvement in LDH/VDH and lleDH/VDH activity ratios were selected. Point mutations in the Vdh protein encoded by the vdh gene in these three mutants were: V319A or I323T or R330H.

TABLE 3
Fold change in the ratio of LDH:VDH and IIeDH:VDH activity of
Vdh mutant proteins compared to the wildtype Vdh protein.

| Point mutation | Amino acid | | |
|---|---|---|---|
| in Vdh protein | sequence | R LDH:VDH | R IIeDH:VDH |
| wildtype | SEQ ID NO: 2 | Reference 1 | Reference 2 |
| V319A | SEQ ID NO: 7 | + | + |
| I323T | SEQ ID NO: 9 | + | + |
| R330H | SEQ ID NO: 11 | + | + |

The activity ratios R LDH:VDH and R lleDH:VDH of the wildtype Vdh protein are referred to as “reference 1” and “reference 2”, respectively. The symbol “+” indicates an increase in the ratio value by a factor greater than 1 but less than 2 as compared to the ratio value of the corresponding “reference”.

As shown in Table 3, each mutation, V319A, I323T, or R330H, increases the LDH:VDH and lleDH:VDH activity ratio by a factor up to 2 as compared to the activity ratio of the Vdh wildtype protein. Thus, a single mutation in Vdh is sufficient to reduce the difference between VDH and LDH or VDH and lleDH activities.

The effect of the V319A mutation was particularly surprising as Baker et al, 1997 and Wang et al, 2001 both failed to convert the Clostridium symbiosum glutamate dehydrogenase into leucine dehydrogenase by substituting the serine at position 380 with valine or alanine (position 380 in the glutamate dehydrogenase described by Baker et al, 1997 or Wang et al., 2001 is equivalent to position 319 of Vdh and position 294 of Ldh).

### Example 2: Evaluation of Cooperative Effect of vdh Mutation Sites

In order to further improve the ratio between LDH or lleDH and VDH activities, the V319A, I323T, and R330H mutations were combined. Using appropriate oligonucleotides and templates, different vdh mutant fragments were amplified by PCR. PCR products were cloned into plasmid 1 (instead of the optimized vdh gene coding for wildtype Vdh protein) according to protocol 3 and E. coli DH5alpha commercial competent cells were transformed with the ligation products. The strains expressing different mutant Vdh proteins were grown and the ratio of LDH:VDH and lleDH:VDH activities was determined and compared to wildtype Vdh.

TABLE 4
Fold change of in the ratio of LDH:VDH and IIeDH:VDH activity
of Vdh mutant proteins compared to the wildtype Vdh protein.

| Point mutation | Amino acid | | |
|---|---|---|---|
| in Vdh protein | sequence | R LDH:VDH | R IIeDH:VDH |
| wildtype | SEQ ID NO: 2 | Reference 1 | Reference 2 |
| V319A/I323T | SEQ ID NO: 13 | + | + |
| V319A/R330H | SEQ ID NO: 15 | + | + |
| I323T/R330H | SEQ ID NO: 17 | + | + |
| V319A/I323T/ | SEQ ID NO: 19 | + | + |
| R330H | | | |

The activity ratios R LDH:VDH and R lleDH:VDH of the wildtype Vdh protein are referred to as “reference 1” and “reference 2”, respectively. The symbol “+” indicates an increase in the ratio value by a factor greater than 1 but less than 2 as compared to the ratio value of the corresponding “reference”.

As shown in Table 4, each combination of mutations increases the value of the LDH:VDH and lleDH:VDH activity ratio by a factor of up to 2 relative to the ratio value of the wildtype protein, but there is little or no cooperative effect between mutations as the ratio values of combined mutants do not exceed, or only slightly exceed, the ratio values obtained with single mutations.

### Example 3: Conservation of vdh Mutation Site Effect Among Amino Acid Dehydrogenases and Cooperative Effect of Mutation Sites in the Ldh Protein

### Conservation of vdh Mutation Site Effect

To assess whether the effect of mutations V319A, I323T, and R330H is conserved among amino acid dehydrogenases, these three mutations were tested in the leucine dehydrogenase Ldh of Thermoactinomyces intermedius (SEQ ID NO: 21, Uniprot Q60030) encoded by the ldh gene (SEQ ID NO: 20).

The ldh gene was synthetically synthesized with the codon usage being optimized to E. coli (SEQ ID NO: 22). Using protocol 3, the optimized ldh gene having the sequence of SEQ ID NO: 22, coding for the wildtype Ldh protein, was cloned under the PR promoter together with the cl857 allele of the thermosensitive repressor of lambda phage (amplified from the pFC1 vector) into the pCL1920 vector, giving the “plasmid 2” plasmid.

By aligning the Vdh (SEQ ID NO: 2) and Ldh (SEQ ID NO: 21) proteins encoded by vdh and ldh genes, respectively, positions equivalent to V319, I323, and R330 in Vdh could be determined in the Ldh protein: V319 in Vdh corresponds to V294 in Ldh, 1323 in Vdh corresponds to L298 in Ldh, and R330 in Vdh corresponds to R305 in Ldh.

By using appropriate oligonucleotides and templates, ldh mutant genes coding for proteins with mutations V294A, L298T, or R305H, were amplified using protocol 4, cloned into plasmid 2 (instead of the optimized ldh gene coding for the wildtype Ldh protein) using protocol 3 and E. coli DH5alpha competent cells were transformed. The strains expressing different mutated genes leading to the expression of Ldh mutant proteins were grown and ratios of LDH:VDH and lleDH:VDH activities were determined and compared to wildtype Ldh using protocol 6.

TABLE 5
Fold change in the ratio of LDH:VDH and IIeDH:VDH activity of
Ldh mutant proteins compared to the wildtype Ldh protein.

| Point mutation | Amino acid | | |
|---|---|---|---|
| in Ldh protein | sequence | R LDH:VDH | R IIeDH: VDH |
| wildtype | SEQ ID NO: 21 | Reference 3 | Reference 4 |
| V294A | SEQ ID NO: 24 | ++ | + |
| L298T | SEQ ID NO: 26 | ++ | + |
| R305H | SEQ ID NO: 28 | ++ | + |

The activity ratios R LDH:VDH and R lleDH:VDH of the wildtype Ldh protein are referred to as “reference 3” and “reference 4”, respectively. The symbol “+” indicates an increase in the ratio value by a factor greater than 1 but less than 2, the symbol “++” indicates an increase by a factor between 2 and 3, as compared to the ratio value of the corresponding “reference”.

As shown in Table 5, each mutation, V294A, L298T, or R305H, has an effect on the ratio of activities, showing that one mutation in Ldh is also sufficient to reduce the difference between VDH and LDH or VDH and lleDH activities and that the effect of each of these three mutations is conserved among amino acid dehydrogenases. More precisely, the value of the activity ratio is increased by a factor between 2 and 3 for LDH:VDH, and by a factor of up to 2 for lleDH:VDH, relative to the ratio values of the Ldh wildtype protein.

### Cooperative Effect of Mutation Sites in Ldh Protein

In order to evaluate if the three selected mutations have a cooperative effect in the Ldh protein, the V294A, L298T, and R305H mutations were combined. Using protocol 4 and appropriate oligonucleotides and templates, different ldh mutant fragments were amplified by PCR. PCR products were cloned into plasmid 2 (instead of the optimized/dh gene coding for the wildtype Ldh protein) using protocol 3 and E. coli DH5alpha commercial competent cells were transformed with the ligation products. The strains expressing different mutant Ldh proteins were grown and the ratio of LDH:VDH and lleDH:VDH activities were determined and compared to wildtype Ldh.

TABLE 6
Fold change in the ratio of LDH:VDH and IIeDH:VDH activity of
Ldh mutant proteins compared to the wildtype Ldh protein.

| Point mutation | Amino acid | | |
|---|---|---|---|
| in Ldh protein | sequence | R LDH:VDH | R IIeDH:VDH |
| wildtype | SEQ ID NO: 21 | Reference 3 | Reference 4 |
| V294A/L298T | SEQ ID NO: 30 | +++ | ++ |
| V294A/R305H | SEQ ID NO: 32 | +++ | ++ |
| L298T/R305H | SEQ ID NO: 34 | +++ | ++ |
| V294A/L298T/ | SEQ ID NO: 36 | +++ | ++ |
| R305H | | | |

The activity ratios R LDH:VDH and R lleDH:VDH of the wildtype Ldh protein are referred to as “reference 3” and “reference 4”, respectively. The symbol “++” indicates an increase by a factor between 2 and 3 and “+++” indicates an increase by a factor greater than 3, as compared to the ratio value of the corresponding “reference”.

As shown in Table 6, each combination of mutations increases the value of the activity ratio by a factor greater than 3 for LDH:VDH, and by a factor between 2 and 3 for lleDH:VDH, relative to the ratio values of the corresponding wildtype protein. Thus, the ratio values of combined mutants exceed the ratio values of the single mutations, showing a cooperative effect between mutations in Ldh, and once more showing a greater effect in the Ldh protein when compared to the Vdh protein.

### Example 4: Application of Vdh and Ldh Mutants to L-Leucine and L-Isoleucine Fermentation

### Construction of Strain 2

Strain 2 was an E. coli MG1655 strain obtained by sequentially:
knocking out the lactate dehydrogenase (ldhA gene), the alcohol dehydrogenase (adhE gene) and the methylglyoxal synthase (mgsA gene),
replacing the 2-isopropylmalate synthase (leuA gene) and the acetohydroxy acid synthase I small regulatory subunit (ilvN gene) by leucine and valine feedback resistant (FBR) proteins respectively (substitution G1435T for leuA* FBR allele (SEQ ID NO: 45)—U.S. Pat. No. 6,403,342; substitutions G59A, C60T, T62A, A63C, A64T, and G66C for ilvN* FBR allele (SEQ ID NO: 49)—Park et al., 2012), and native promoters were replaced by an artificial Ptrc promoter (Brosius et al, 1985),
   according to protocols 1 and 2.

### Construction of Strain 3

The chromosomal modifications of strain 3 were those described in Park et al., 2012.

Briefly, strain 3 was an E. coli MG1655 obtained by sequentially:
knocking out the diaminopimelate decarboxylase (lysA gene), the homoserine O-succinyltransferase (metA gene), the threonine dehydrogenase (tdh gene), the transcriptional regulator “Isocitrate lyase Regulator” (icIR gene),
replacing aspartokinases I and III (thrA and lysC genes, respectively) and the threonine dehydratase (ilvA gene) by threonine, lysine, and isoleucine feedback resistant proteins respectively (substitution C1034T for thrA* FBR allele (SEQ ID NO: 61), substitution C1055T for lysC*FBR allele (SEQ ID NO: 65) and substitutions C1339T, G1341T, C1351G, and T1352C for ilvA* FBR allele (SEQ ID NO: 69)), and native promoters were replaced by an artificial Ptrc promoter,
overexpressing the ketol-acid reductoisomerase (ilvC gene), the phosphoenolpyruvate carboxylase (ppc gene) and the “Leucine-responsive regulatory protein” transcriptional regulator (lrp gene) under an artificial Ptrc promoter on chromosome, according to protocols 1 and 2.

### Construction of Strains 4 to 27

### Construction of Leucine Producer Strains

According to protocol 3, the dihydroxy-acid dehydratase (ilvD gene), the ketol-acid reductoisomerase (ilvC gene), the acetohydroxy acid synthase I (both subunits encoded by ilvBN genes, with ilvN* FBR allele), the 2-isopropylmalate synthase (leuA* FBR allele), the 3-isopropylmalate dehydrogenase (leuB gene), and the 3-isopropylmalate dehydratase subunits (leuCD genes) were organized into an operon downstream of valine dehydrogenase (optimized vdh allele coding for the wildtype Vdh protein) on plasmid 1, giving rise to plasmid 3. The vdh gene, which was codon-optimized for E. coli expression, originated from Streptomyces aureofaciens while other genes originated from E. coli. Plasmid 3 was introduced into strain 2, giving rise to strain 4 as mentioned in Table 7. The optimized vdh allele (coding for wildtype Vdh protein) of plasmid 3 was replaced with different vdh* alleles using protocol 3 and each plasmid was introduced individually into strain 2, giving rise to strains 5 to 11, described in Table 7.

Alternatively, the ilvD, ilvC, ilvBN*, leuA*BCD genes from E. coli were organized into an operon downstream of leucine dehydrogenase, (the ldh gene, which was codon-optimized for E. coli expression, originated from Thermoactinomyces intermedius while other genes originated from E. coli) on plasmid 2 using protocol 3, giving rise to plasmid 4.

Plasmid 4 was introduced into strain 2, giving rise to strain 12 as mentioned in Table 7. The optimized ldh allele (coding for the wildtype Ldh protein) of plasmid 4 was replaced with different ldh* alleles using protocol 3 and each plasmid was introduced individually into strain 2, giving rise to strains 13 to 19, described in Table 7.

### Construction of Isoleucine Producer Strains

The dihydroxy-acid dehydratase and the threonine deaminase (encoded by the ilvD and ilvA* genes respectively, with ilvA* FBR allele), the ketol-acid reductoisomerase (ilvC gene), the acetolactate synthase III (both subunits encoded by ilvIH genes, with valine and isoleucine FBR ilvH* allele having substitutions G41A and C50T (SEQ ID NO: 89)), the aspartate kinase/homoserine dehydrogenase 1, the homoserine kinase, and the threonine synthase (thrA, thrB and thrC genes, respectively, with thrA* FBR allele) and the branched chain amino acid export (ygaZ and ygaH genes) were organized into an operon downstream of the leucine dehydrogenase (optimized ldh allele coding for the wildtype Ldh protein) on plasmid 2 using protocol 3, giving rise to plasmid 5.

The optimized ldh allele (coding for the wildtype Ldh protein) of plasmid 5 was replaced with different ldh* alleles using protocol 3 and each plasmid was introduced individually into strain 3, giving rise to strains 20 to 27, described in Table 7.

TABLE 7
Producing strains obtained with different
mutated Vdh and Ldh proteins.

| Point mutation | into Strain | Point mutation | | |
|---|---|---|---|---|
| in Vdh protein | 2 | in Ldh protein | into Strain 2 | into Strain 3 |
| wildtype | Strain 4 | wildtype | Strain 12 | Strain 20 |
| V319A | Strain 5 | V294A | Strain 13 | Strain 21 |
| I323T | Strain 6 | L298T | Strain 14 | Strain 22 |
| R330H | Strain 7 | R305H | Strain 15 | Strain 23 |
| V319A/I323T | Strain 8 | V294A/L298T | Strain 16 | Strain 24 |
| V319A/R330H | Strain 9 | V294A/R305H | Strain 17 | Strain 25 |
| I323T/R330H | Strain 10 | L298T/R305H | Strain 18 | Strain 26 |
| V319A/I323T/ | Strain 11 | V294A/L298T/ | Strain 19 | Strain 27 |
| R330H | | R305H | | |

Improvement of Leucine Production with Vdh or Ldh Mutant Proteins

Strains 4 to 19 were grown according to protocol 7. Leucine and valine yields were measured.

TABLE 8
Leucine yield and ratio of leucine:valine titers for the strains
4 to 11 with mutated Vdh proteins compared to the wildtype Vdh.

| Point mutation | | Leucine | Leucine titer:Valine |
|---|---|---|---|
| of Vdh protein | Strain | yield (g/g) | titer ((g/L)/(g/L)) |
| wildtype | Strain 4 | Reference 5 | Reference 6 |
| V319A | Strain 5 | + | ++ |
| I323T | Strain 6 | ≈ | ++ |
| R330H | Strain 7 | ≈ | ++ |
| V319A/I323T | Strain 8 | + | ++ |
| V319A/R330H | Strain 9 | ≈ | ++ |
| I323T/R330H | Strain 10 | ≈ | ++ |
| V319A/I323T/R330H | Strain 11 | ≈ | ++ |

Leucine yield and the ratio of leucine:valine titers of strain 4 carrying the wildtype Vdh protein are referred to as “reference 5” and “reference 6”, respectively. The symbol “≈” indicates no increase, “+” indicates an increase between 0% and 10%, and the symbol “++” an increase between 10% and 20%, as compared to the corresponding reference values of strain 4.

As shown in Table 8, all strains show an increased ratio of leucine titer:valine titer, which corroborates activity ratio data of Vdh mutant proteins. The increase in the ratio of leucine titer:valine titer is highly advantageous as downstream purification of leucine is facilitated. Strains 5 and 8 furthermore advantageously show an increase in leucine yield.

TABLE 9
Leucine yield and ratio of leucine:valine titers for strains 12
to 19 with mutated Ldh proteins compared to the wildtype Ldh.

| Point mutation | | Leucine | Leucine titer:Valine |
|---|---|---|---|
| in Ldh protein | Strain | yield (g/g) | titer ((g/L)/(g/L)) |
| wildtype | Strain 12 | Reference 7 | Reference 8 |
| V294A | Strain 13 | ++ | ++ |
| L298T | Strain 14 | + | ++ |
| R305H | Strain 15 | + | + |
| V294A/L298T | Strain 16 | ++ | +++ |
| V294A/R305H | Strain 17 | ++ | +++ |
| L298T/R305H | Strain 18 | + | +++ |
| V294A/L298T/R305H | Strain 19 | + | +++ |

Leucine yield and the ratio of leucine:valine titers of strain 12 carrying the wildtype Ldh protein are referred to as “reference 7” and “reference 8”, respectively. The symbol “˜” indicates no increase, “+” indicates an increase between 0% and 10%, the symbol “++” an increase between 10% and 20%, and “+++” an increase greater than 20%, as compared to the corresponding reference values of strain 12.

As can be seen in Table 9, all strains show an increased ratio of leucine titer:valine titer, which is further increased in the presence of Ldh having multiple mutations, corroborating activity ratio data obtained for the Ldh mutant proteins. The increase in the ratio of leucine titer:valine titer is highly advantageous as downstream purification of leucine is facilitated. All strains furthermore advantageously show an increase in leucine yield.

Improvement of Isoleucine Production with Mutation Sites in the Ldh Protein

Strains 20 to 27 were grown according to protocol 7. Isoleucine and valine yields produced were measured.

The effect of the mutated Ldh protein (strains 21 to 27) on isoleucine yield and the ratio of isoleucine:valine titers when compared to strain 20 were similar to those obtained with leucine producing strains. The increase in the ratio of isoleucine titer:valine titer is highly advantageous as downstream purification of isoleucine is facilitated.

### REFERENCES

Anderson, (1946), Proc. Natl. Acad. Sci. USA., 32:120-128.
Baker et al, (1997) Biochemistry, 36(51): 16109-16115
Bantscheff et al., (2007), Analytical and Bioanalytical Chemistry, vol. 389(4): 1017-1031.
Brosius et al, (1985), J Biol Chem, 260(6): 3539-3541
Burnette, (1981), Analytical Biochemistry, 112(2): 195-203.
Datsenko and Wanner, (2000), Proc Natl Acad Sci USA., 97:6640-6645.
Davis & Olsen., (2011), Mol. Biol. Evol., 28(1): 211-221.
Dayhoff et al. (1978), “A model of evolutionary change in proteins,” in “Atlas of Protein Sequence and Structure,” Vol. 5, Suppl. 3 (ed. M. O. Dayhoff), p. 345-352. Natl. Biomed. Res. Found., Washington, D.C.
Deml et al., (2011), J. Virol., 75(22): 10991-11001.
Durbin et al., (1998), Biological Sequence Analysis, Cambridge University Press.
Engvall and Perlman (1981), Immunochemistry, 8: 871-874.
Graf et al., (2000), J. Virol., 74(22): 10/22-10826.
Henikoff and Henikoff (1992), Proc. Natl. Acad. Sci. USA, 89: 10915-10919
Lerner & Inouye, (1990), Nucleic Acids Research, 18(15): 4631
Leuchtenberger, et al, (2005) Appl. Microbiol. Biotechnol. 69, 1-8
Mermet-Bouvier & Chauvat, (1994), Current Microbiology, 28: 145-148
Miller, (1992) “A Short Course in Bacterial Genetics: A Laboratory Manual and Handbook for Escherichia coli and Related Bacteria”, Cold Spring Harbor Laboratory Press, Cold Spring Harbor, N.Y.
Needleman and Wunsch (1970), J. Mol. Biol., 48(3), 443-453.
Ohashima and Soda (1979), Eur J Biochem, 100(1): 29-30
Park et al., (2012), ACS synthetic biology, 1(11): 532-540
Park and Lee, (2010), Appl Microbiol Biotechnol 85(3): 491-506
Schaefer et al., (1999), Anal. Biochem. 270: 88-96.
Wang et al, (2001), European journal of biochemistry, 268(22): 5791-5799
Yamamoto et al, (2017), Adv Biochem Eng Biotechnol. 159:103-128.

1. A modified dehydrogenase, wherein one or more amino acid residues of the dehydrogenase are modified as compared to an unmodified dehydrogenase, and wherein the modified dehydrogenase has an increased ratio of leucine dehydrogenase or isoleucine dehydrogenase activity to valine dehydrogenase activity as compared to the unmodified dehydrogenase.

2. The modified dehydrogenase of claim 1, wherein the dehydrogenase is a leucine or valine dehydrogenase.

3. The modified dehydrogenase of claim 1, wherein the dehydrogenase has at least 95% sequence similarity to the dehydrogenase having the sequence of SEQ ID NO: 2 and wherein the dehydrogenase comprises an alanine at the position corresponding to position 319, a threonine at the position corresponding to position 323, and/or a histidine at the position corresponding to position 330 of SEQ ID NO: 2.

4. The modified dehydrogenase claim 1, wherein the dehydrogenase is selected from the group consisting of:
Vdh* (V319A) of sequence SEQ ID NO: 7,
Vdh* (I323T) of sequence SEQ ID NO: 9,
Vdh* (R330H) of sequence SEQ ID NO: 11,
Vdh* (V319A/I323T) of sequence SEQ ID NO: 13,
Vdh* (V319A/R330H) of sequence SEQ ID NO: 15,
Vdh* (I323T/R330H) of sequence SEQ ID NO: 17, and
Vdh* (V319A/I323T/R330H) of sequence SEQ ID NO: 19.

5. The modified dehydrogenase of claim 1, wherein the dehydrogenase has at least 95% sequence similarity to the dehydrogenase having the sequence of SEQ ID NO: 21 and wherein the dehydrogenase comprises an alanine at the position corresponding to position 294, a threonine at the position corresponding to position 298, and/or a histidine at the position corresponding to position 305 of SEQ ID NO: 21.

6. The modified dehydrogenase of claim 5, wherein the dehydrogenase is selected from the group consisting of:
Ldh* (V294A) of sequence SEQ ID NO: 24,
Ldh* (L298T) of sequence SEQ ID NO: 26,
Ldh* (R305H) of sequence SEQ ID NO: 28,
Ldh* (V294A/L298T) of sequence SEQ ID NO: 30,
Ldh* (V294A/R305H) of sequence SEQ ID NO: 32,
Ldh* (L298T/R305H) of sequence SEQ ID NO: 34, and
Ldh* (V294A/L298T/R305H) of sequence SEQ ID NO: 36.

7. The dehydrogenase of claim 1, wherein the dehydrogenase is coded by a Vdh* gene having the sequence of one of SEQ ID NOs: 6, 8, 10, 12, 14, 16, 18 or an ldh* gene having the sequence of one of SEQ ID NOs: 23, 25, 27, 29, 31, 33, 35.

8. An Escherichia coli microorganism genetically modified for the increased production of a branched chain amino acid selected from leucine and isoleucine, wherein the microorganism further comprises the expression of at least one heterologous gene coding a modified dehydrogenase according to claim 1.

9. The microorganism for the production of leucine of claim 8, comprising a deletion of at least one of the following genes: adhE, ldhA, and mgsA, and an overexpression of at least one of the following genes: ilvC, ilvD, ilvBN*, and leuA*BCD, wherein the ilvN* gene codes for a polypeptide having the sequence of SEQ ID NO: 50 and the leuA* gene codes for a polypeptide having the sequence of SEQ ID NO: 46.

10. The microorganism for the production of isoleucine of claim 8, comprising a deletion of at least one of the following genes: lysA, metA, tdh and iclR and an overexpression of at least one of the following genes: ilvDA*, ilvC, ilvH*, Irp, thrA*BC, lysC*, ppc and ygaZH, wherein the ilvA* gene codes for a polypeptide having the sequence of SEQ ID NO: 70, wherein the lysC* gene codes for a polypeptide having the sequence of SEQ ID NO: 66 and wherein the ilvH* gene codes for a polypeptide having the sequence of SEQ ID NO: 90.

11. The microorganism of claim 8, wherein the microorganism has been genetically modified to be able to utilize sucrose as a carbon source, preferably wherein said microorganism further comprises the overexpression of:
the heterologous cscBKAR genes of E. coli EC3132, or
the heterologous scrKYABR genes of Salmonella sp.

12. A method for the fermentative production of a branched chain amino acid selected from leucine and isoleucine, comprising the steps of:
a) culturing, under fermentative conditions, an Escherichia coli microorganism according to claim 8, in a culture medium comprising a carbohydrate as a source of carbon; and
b) recovering the branched chain amino acid from the culture medium.

13. The method according to claim 12, wherein the source of carbon is glucose and/or sucrose.

14. The method according to claim 12, further comprising a step c) of purifying the branched chain amino acid recovered in step b).

15. The microorganism according to claim 11, wherein said microorganism genetically modified to be able to utilize sucrose as a carbon source further comprises the overexpression of:
the heterologous cscBKAR genes of E. coli EC3132, or
the heterologous scrKYABR genes of Salmonella sp.


SEQUENCE LISTING (116 sequences)

SEQ ID NO: 1 - DNA, 1158 bp, Streptomyces aureofaciens
  source 1..1158: mol_type = genomic DNA
gtgaccgacgtacagaccccggccacgcaccccgcacccggcgtgctcagcaggatcttccgcaccgagcaggacggcgcccccggcgacggccacgagcaggtcgtcctctgccacgaccgcagctccgggctcaaggcgatcatcgccatccactccaccgccctcggccccgccctcggcggcacccgcttcttcccgtacgcctccgaggaggcggccctggaggacgcgctgaacctctcccgcgggatgtcctacaagaacgcgctcgccggcctcgacctcggcggcggcaaggccgtcatcatcggtgacccgaacaaggacaagaacgaggcgatgctccgggcctacggccgcttcgtcgagtccctgcgcggccgctacatcaccgcctgcgacgtgggcacctacgtccaggacatggacgtcatcgcgcgcgagaccgagttcgtgaccggccgctcgcccgagcacggcggcgccggcgactcgtcgatcctgaccgccttcggcgtcttccagggcatgcgcgcctccgcccaggcccgttggggccagcccaccctgcgcggcaagcgggtcggcgtcgcgggcgtcggcaaggtcggccactacctggtcggccacctggtcgcggacggcgccaccgtggtcgtcaccgaccccttcgaggccgccgtcaaccgcgtgcgcgcggcccacccggaggtggaggtggtcgccgacaccaccgccctgctccaggccaagctggacgtctacgccccctgcgccctcggcggcgcgctgaccgaccccgtggtcgcggcgctcagcgagttcggcacctccatcgtctgcggcgcggccaacaaccagctggcccacccgggcgtcgagaaggacctgtccgaccgcggcatcctctacgcccccgactacctggtgaactccggcggcgtgatccaggtcgccgacgagatcgacggcttcaacttcgagcgggccaagaacaaggccaccaagatcttcgacaccaccctggagatcttcacccgggcagccgccgacggcgtcccgccggcggtcgccgccgaccgcctggccgagaagcggatgcgcgagatcagcgcgctgcgcacggtcctgctgccgggcgcccgccgcggctga

SEQ ID NO: 2 - protein, 385 aa, Streptomyces aureofaciens
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQVADEIDGFNFERAKNKATKIFDTTLEIFTRAAADGVPPAVAADRLAEKRMREISALRTVLLPGARRG

SEQ ID NO: 3 - DNA, 1158 bp, synthetic construct
  misc_feature 1..1158: note = vdh gene with optimised codons
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagtggcggacgagattgatggcttcaactttgaacgtgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 4 - DNA, 714 bp, lambda phage
  source 1..714: mol_type = genomic DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 5 - protein, 237 aa, lambda phage
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 6 - DNA, 1158 bp, synthetic construct
  misc_feature 1..1158: note = (codon usage) optimised gene for Vdh* (V319A)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagcggcggacgagattgatggcttcaactttgaacgtgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 7 - protein, 385 aa, synthetic construct
  REGION 1..385: note = Vdh* (V319A)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQAADEIDGFNFERAKNKATKIFDTTLEIFTRAAADGVPPAVAADRLAEKRMREISALRTVLLPGARRG

SEQ ID NO: 8 - DNA, 1158 bp, synthetic construct
  misc_feature 1..1158: note = (codon usage) optimised gene for Vdh* (I323T)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagtggcggacgagactgatggcttcaactttgaacgtgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 9 - protein, 385 aa, synthetic construct
  REGION 1..385: note = Vdh* (I323T)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQVADETDGFNFERAKNKATKIFDTTLEIFTRAAADGVPPAVAADRLAEKRMREISALRTVLLPGARRG

SEQ ID NO: 10 - DNA, 1158 bp, synthetic construct
  misc_feature 1..1158: note = (codon usage) optimised gene for Vdh* (R330H)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagtggcggacgagattgatggcttcaactttgaacatgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 11 - protein, 385 aa, synthetic construct
  REGION 1..385: note = Vdh* (R330H)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQVADEIDGFNFEHAKNKATKIFDTTLEIFTRAAADGVPPAVAADRLAEKRMREISALRTVLLPGARRG

SEQ ID NO: 12 - DNA, 1158 bp, synthetic construct
  misc_feature 1..1158: note = (codon usage) optimised gene for Vdh* (V319A/I323T)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagcggcggacgagactgatggcttcaactttgaacgtgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 13 - protein, 385 aa, synthetic construct
  REGION 1..385: note = Vdh* (V319A/I323T)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQAADETDGFNFERAKNKATKIFDTTLEIFTRAAADGVPPAVAADRLAEKRMREISALRTVLLPGARRG

SEQ ID NO: 14 - DNA, 1158 bp, synthetic construct
  misc_feature 1..1158: note = (codon usage) optimised gene for Vdh* (V319A/R330H)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagcggcggacgagattgatggcttcaactttgaacatgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 15 - protein, 385 aa, synthetic construct
  REGION 1..385: note = Vdh* (V319A/R330H)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQAADEIDGFNFEHAKNKATKIFDTTLEIFTRAAADGVPPAVAADRLAEKRMREISALRTVLLPGARRG

SEQ ID NO: 16 - DNA, 1158 bp, synthetic construct
  misc_feature 1..1158: note = (codon usage) optimised gene for Vdh* (I323T/R330H)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagtggcggacgagactgatggcttcaactttgaacatgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 17 - protein, 385 aa, synthetic construct
  REGION 1..385: note = Vdh* (I323T/R330H)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQVADETDGFNFEHAKNKATKIFDTTLEIFTRAAADGVPPAVAADRLAEKRMREISALRTVLLPGARRG

SEQ ID NO: 18 - DNA, 1158 bp, synthetic construct
  misc_feature 1..1158: note = (codon usage) optimised gene for Vdh* (V319A/I323T/R330H)
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagcggcggacgagactgatggcttcaactttgaacatgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 19 - protein, 385 aa, synthetic construct
  REGION 1..385: note = Vdh* (V319A/I323T/R330H)
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQAADETDGFNFEHAKNKATKIFDTTLEIFTRAAADGVPPAVAADRLAEKRMREISALRTVLLPGARRG

SEQ ID NO: 20 - DNA, 1101 bp, Thermoactinomyces intermedius
  source 1..1101: mol_type = genomic DNA
atgaaaatcttcgattacatggaaaaatatgattatgaacaactcgtcatgtgccaagataaagagtccggcctcaaagccatcatttgtatccatgttaccaccctggggccggctctgggtggtatgcgcatgtggacttacgcttcggaagaagaagccatcgaggatgcgctgcgcctgggaagagggatgacttacaaaaatgcagctgccggcttaaacctcggcggcggaaaaacggtgatcatcggcgatccgagaaaagacaaaaacgaagccatgttccgtgctttgggacgctttatccaaggattgaacggacggtacatcacggcggaagacgtgggaaccactgtggaagacatggatatcattcatgaggaaacccgttacgtgacaggcgtatcgcccgctttcggttccagcggcaacccttcaccggtgacggcttacggcgtataccgtggaatgaaagcggctgcaaaagaagctttcggcgatgactcactcgaaggcaaagtggtggctgttcaaggagtgggacatgttgcttacgaattatgtaagcacttgcataatgaaggggccaaattgattgtgaccgacatcaacaaagaaaatgcagaccgtgccgtgcaagaatttggtgccgaatttgttcatccggacaaaatctatgatgtggaatgcgacatttttgctccttgtgcgctcggggcgatcattaatgacgaaaccattgaacgcttgaaatgcaaagtggtggccggttcggcaaacaatcagctgaaagaagagcgccatggaaaaatgcttgaagaaaaaggaatcgtctatgcgccggattatgtgatcaatgccggaggcgtgatcaatgtcgccgatgaattgctgggttataaccgtgagcgtgcgatgaaaaaagtggaaggcatttacgacaaaatcctgaaagtgtttgagatcgcaaaacgggacggcattccgagctatctcgcagcagaccgcatggcggaagaacggattgaaatgatgcgcaaaacaagaagcacatttttgcaagatcaacgcaacctgatcaattttaacaacaaataa

SEQ ID NO: 21 - protein, 366 aa, Thermoactinomyces intermedius
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINVADELLGYNRERAMKKVEGIYDKILKVFEIAKRDGIPSYLAADRMAEERIEMMRKTRSTFLQDQRNLINFNNK

SEQ ID NO: 22 - DNA, 1101 bp, synthetic construct
  misc_feature 1..1101: note = ldh gene with optimised codons
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgtggccgatgaactcctgggctataaccgtgaacgcgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 23 - DNA, 1101 bp, synthetic construct
  misc_feature 1..1101: note = (codon usage) optimised gene for Ldh* (V294A)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgcggccgatgaactcctgggctataaccgtgaacgcgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 24 - protein, 366 aa, synthetic construct
  REGION 1..366: note = Ldh* (V294A)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINAADELLGYNRERAMKKVEGIYDKILKVFEIAKRDGIPSYLAADRMAEERIEMMRKTRSTFLQDQRNLINFNNK

SEQ ID NO: 25 - DNA, 1101 bp, synthetic construct
  misc_feature 1..1101: note = (codon usage) optimised gene for Ldh* (L298T)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgtggccgatgaaactctgggctataaccgtgaacgcgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 26 - protein, 366 aa, synthetic construct
  REGION 1..366: note = Ldh* (L298T)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINVADETLGYNRERAMKKVEGIYDKILKVFEIAKRDGIPSYLAADRMAEERIEMMRKTRSTFLQDQRNLINFNNK

SEQ ID NO: 27 - DNA, 1101 bp, synthetic construct
  misc_feature 1..1101: note = (codon usage) optimised gene for Ldh* (R305H)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgtggccgatgaactcctgggctataaccgtgaacatgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 28 - protein, 366 aa, synthetic construct
  REGION 1..366: note = Ldh* (R305H)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINVADELLGYNREHAMKKVEGIYDKILKVFEIAKRDGIPSYLAADRMAEERIEMMRKTRSTFLQDQRNLINFNNK

SEQ ID NO: 29 - DNA, 1101 bp, synthetic construct
  misc_feature 1..1101: note = (codon usage) optimised gene for Ldh* (V294A/L298T)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgcggccgatgaaactctgggctataaccgtgaacgcgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 30 - protein, 366 aa, synthetic construct
  REGION 1..366: note = Ldh* (V294A/L298T)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINAADETLGYNRERAMKKVEGIYDKILKVFEIAKRDGIPSYLAADRMAEERIEMMRKTRSTFLQDQRNLINFNNK

SEQ ID NO: 31 - DNA, 1101 bp, synthetic construct
  misc_feature 1..1101: note = (codon usage) optimised gene for Ldh* (V294A/R305H)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgcggccgatgaactcctgggctataaccgtgaacatgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 32 - protein, 366 aa, synthetic construct
  REGION 1..366: note = Ldh* (V294A/R305H)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINAADELLGYNREHAMKKVEGIYDKILKVFEIAKRDGIPSYLAADRMAEERIEMMRKTRSTFLQDQRNLINFNNK

SEQ ID NO: 33 - DNA, 1101 bp, synthetic construct
  misc_feature 1..1101: note = (codon usage) optimised gene for Ldh* (L298T/R305H)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgtggccgatgaaactctgggctataaccgtgaacatgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 34 - protein, 366 aa, synthetic construct
  REGION 1..366: note = Ldh* (L298T/R305H)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINVADETLGYNREHAMKKVEGIYDKILKVFEIAKRDGIPSYLAADRMAEERIEMMRKTRSTFLQDQRNLINFNNK

SEQ ID NO: 35 - DNA, 1101 bp, synthetic construct
  misc_feature 1..1101: note = (codon usage) optimised gene for Ldh* (V294A/L298T/R305H)
  source 1..1101: mol_type = other DNA
atgaaaatctttgactacatggaaaagtacgattatgaacagctggttatgtgccaggataaagagtcgggtttaaaagcgatcatttgcattcatgtcaccacgttaggcccggctttgggtggcatgcgtatgtggacgtacgcatcggaagaagaagcgattgaagatgcacttcgccttggtcgtggcatgacgtataagaacgcggcggccggtcttaacttaggcggtggaaagaccgtcatcattggagacccgcgtaaagacaagaacgaagccatgttccgcgcccttggccgttttatccagggtctgaatggacgttatattaccgccgaagacgtcggcactacagttgaggacatggacatcattcatgaggaaacccgttatgttactggggtttcgcccgccttcggcagctcgggaaaccctagcccggtgaccgcttatggcgtgtatcgtgggatgaaagccgccgcgaaggaagcgtttggggatgactctttagagggcaaagtagtagccgtccagggagttggtcatgtggcctacgaactctgtaaacaccttcataacgaaggtgcaaaacttatcgttactgatattaacaaagaaaacgctgatcgcgccgtacaggagtttggcgctgaatttgtgcatccggataaaatctatgacgttgaatgcgatattttcgcgccctgtgcgctgggagcgattattaacgacgaaacgatcgaacgcctcaaatgtaaggttgtcgccggcagcgccaataaccagttaaaagaggaacgccatggcaagatgctcgaagagaagggtatcgtctacgccccggattatgtgattaacgcgggcggcgtgattaacgcggccgatgaaactctgggctataaccgtgaacatgccatgaagaaagtcgaggggatttatgacaaaattctgaaagtctttgagatcgccaaacgtgatgggatcccttcctacctggccgcggaccgcatggcggaagaacgcattgaaatgatgcgcaaaacgcgctcgacattcttacaggaccagcgcaatctgattaattttaataacaaataa

SEQ ID NO: 36 - protein, 366 aa, synthetic construct
  REGION 1..366: note = Ldh* (V294A/L298T/R305H)
MKIFDYMEKYDYEQLVMCQDKESGLKAIICIHVTTLGPALGGMRMWTYASEEEAIEDALRLGRGMTYKNAAAGLNLGGGKTVIIGDPRKDKNEAMFRALGRFIQGLNGRYITAEDVGTTVEDMDIIHEETRYVTGVSPAFGSSGNPSPVTAYGVYRGMKAAAKEAFGDDSLEGKVVAVQGVGHVAYELCKHLHNEGAKLIVTDINKENADRAVQEFGAEFVHPDKIYDVECDIFAPCALGAIINDETIERLKCKVVAGSANNQLKEERHGKMLEEKGIVYAPDYVINAGGVINAADETLGYNREHAMKKVEGIYDKILKVFEIAKRDGIPSYLAADRMAEERIEMMRKTRSTFLQDQRNLINFNNK

SEQ ID NO: 37 - DNA, 990 bp, Escherichia coli
  source 1..990: mol_type = genomic DNA
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 38 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 39 - DNA, 2676 bp, Escherichia coli
  source 1..2676: mol_type = genomic DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 40 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 41 - DNA, 459 bp, Escherichia coli
  source 1..459: mol_type = genomic DNA
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 42 - protein, 152 aa, Escherichia coli [sequence omitted]

SEQ ID NO: 43 - DNA, 1572 bp, Escherichia coli
  source 1..1572: mol_type = genomic DNA
atgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctgggtcaggtggatatcgtcgctaactacaacggtcgccgcttccacggcgtcggcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtgtga

SEQ ID NO: 44 - protein, 523 aa, Escherichia coli
MSQQVIIFDTTLRDGEQALQASLSVKEKLQIALALERMGVDVMEVGFPVSSPGDFESVQTIARQVKNSRVCALARCVEKDIDVAAESLKVAEAFRIHTFIATSPMHIATKLRSTLDEVIERAIYMVKRARNYTDDVEFSCEDAGRTPIADLARVVEAAINAGATTINIPDTVGYTMPFEFAGIISGLYERVPNIDKAIISVHTHDDLGLAVGNSLAAVHAGARQVEGAMNGIGERAGNCSLEEVIMAIKVRKDILNVHTAINHQEIWRTSQLVSQICNMPIPANKAIVGSGAFAHSSGIHQDGVLKNRENYEIMTPESIGLNQIQLNLTSRSGRAAVKHRMDEMGYKESEYNLDNLYDAFLKLADKKGQVFDYDLEALAFIGKQQEEPEHFRLDYFSVQSGSNDIATAAVKLACGEEVKAEAANGNGPVDAVYQAINRITEYNVELVKYSLTAKGHGKDALGQVDIVANYNGRRFHGVGLATDIVESSAKAMVHVLNNIWRAAEVEKELQRKAQHNENNKETV

SEQ ID NO: 45 - DNA, 1572 bp, synthetic construct
  misc_feature 1..1572: note = leuA* gene
  source 1..1572: mol_type = other DNA
atgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctgggtcaggtggatatcgtcgctaactacaacggtcgccgcttccacggcgtctgcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtgtga

SEQ ID NO: 46 - protein, 523 aa, synthetic construct
  REGION 1..523: note = LeuA* protein
MSQQVIIFDTTLRDGEQALQASLSVKEKLQIALALERMGVDVMEVGFPVSSPGDFESVQTIARQVKNSRVCALARCVEKDIDVAAESLKVAEAFRIHTFIATSPMHIATKLRSTLDEVIERAIYMVKRARNYTDDVEFSCEDAGRTPIADLARVVEAAINAGATTINIPDTVGYTMPFEFAGIISGLYERVPNIDKAIISVHTHDDLGLAVGNSLAAVHAGARQVEGAMNGIGERAGNCSLEEVIMAIKVRKDILNVHTAINHQEIWRTSQLVSQICNMPIPANKAIVGSGAFAHSSGIHQDGVLKNRENYEIMTPESIGLNQIQLNLTSRSGRAAVKHRMDEMGYKESEYNLDNLYDAFLKLADKKGQVFDYDLEALAFIGKQQEEPEHFRLDYFSVQSGSNDIATAAVKLACGEEVKAEAANGNGPVDAVYQAINRITEYNVELVKYSLTAKGHGKDALGQVDIVANYNGRRFHGVCLATDIVESSAKAMVHVLNNIWRAAEVEKELQRKAQHNENNKETV

SEQ ID NO: 47 - DNA, 291 bp, Escherichia coli
  source 1..291: mol_type = genomic DNA
atgcaaaacacaactcatgacaacgtaattctggagctcaccgttcgcaaccatccgggcgtaatgacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaa

SEQ ID NO: 48 - protein, 96 aa, Escherichia coli
MQNTTHDNVILELTVRNHPGVMTHVCGLFARRAFNVEGILCLPIQDSDKSHIWLLVNDDQRLEQMISQIDKLEDVVKVQRNQSDPTMFNKIAVFFQ

SEQ ID NO: 49 - DNA, 291 bp, synthetic construct
  misc_feature 1..291: note = ilvN* gene
  source 1..291: mol_type = other DNA
atgcaaaacacaactcatgacaacgtaattctggagctcaccgttcgcaaccatccggatgacttcacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaa

SEQ ID NO: 50 - protein, 96 aa, synthetic construct
  REGION 1..96: note = IlvN* protein
MQNTTHDNVILELTVRNHPDDFTHVCGLFARRAFNVEGILCLPIQDSDKSHIWLLVNDDQRLEQMISQIDKLEDVVKVQRNQSDPTMFNKIAVFFQ

SEQ ID NO: 51 - DNA, 1263 bp, Escherichia coli
  source 1..1263: mol_type = genomic DNA
atgccacattcactgttcagcaccgataccgatctcaccgccgaaaatctgctgcgtttgcccgctgaatttggctgcccggtgtgggtctacgatgcgcaaattattcgtcggcagattgcagcgctgaaacagtttgatgtggtgcgctttgcacagaaagcctgttccaatattcatattttgcgcttaatgcgtgagcagggcgtgaaagtggattccgtctcgttaggcgaaatagagcgtgcgttggcggcgggttacaatccgcaaacgcaccccgatgatattgtttttacggcagatgttatcgatcaggcgacgcttgaacgcgtcagtgaattgcaaattccggtgaatgcgggttctgttgatatgctcgaccaactgggccaggtttcgccagggcatcgggtatggctgcgcgttaatccggggtttggtcacggacatagccaaaaaaccaataccggtggcgaaaacagcaagcacggtatctggtacaccgatctgcccgccgcactggacgtgatacaacgtcatcatctgcagctggtcggcattcacatgcacattggttctggcgttgattatgcccatctggaacaggtgtgtggtgctatggtgcgtcaggtcatcgaattcggtcaggatttacaggctatttctgcgggcggtgggctttctgttccttatcaacagggtgaagaggcggttgataccgaacattattatggtctgtggaatgccgcgcgtgagcaaatcgcccgccatttgggccaccctgtgaaactggaaattgaaccgggtcgcttcctggtagcgcagtctggcgtattaattactcaggtgcggagcgtcaaacaaatggggagccgccactttgtgctggttgatgccgggttcaacgatctgatgcgcccggcaatgtacggtagttaccaccatatcagtgccctggcagctgatggtcgttctctggaacacgcgccaacggtggaaaccgtcgtcgccggaccgttatgtgaatcgggcgatgtctttacccagcaggaagggggaaatgttgaaacccgcgccttgccggaagtgaaggcaggtgattatctggtactgcatgatacaggggcatatggcgcatcaatgtcatccaactacaatagccgtccgctgttaccagaagttctgtttgataatggtcaggcgcggttgattcgccgtcgccagaccatcgaagaattactggcgctggaattgctttaa

SEQ ID NO: 52 - protein, 420 aa, Escherichia coli
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 53 - DNA, 930 bp, Escherichia coli
  source 1..930: mol_type = genomic DNA
atgccgattcgtgtgccggacgagctacccgccgtcaatttcttgcgtgaagaaaacgtctttgtgatgacaacttctcgtgcgtctggtcaggaaattcgtccacttaaggttctgatccttaacctgatgccgaagaagattgaaactgaaaatcagtttctgcgcctgctttcaaactcacctttgcaggtcgatattcagctgttgcgcatcgattcccgtgaatcgcgcaacacgcccgcagagcatctgaacaacttctactgtaactttgaagatattcaggatcagaactttgacggtttgattgtaactggtgcgccgctgggcctggtggagtttaatgatgtcgcttactggccgcagatcaaacaggtgctggagtggtcgaaagatcacgtcacctcgacgctgtttgtctgctgggcggtacaggccgcgctcaatatcctctacggcattcctaagcaaactcgcaccgaaaaactctctggcgtttacgagcatcatattctccatcctcatgcgcttctgacgcgtggctttgatgattcattcctggcaccgcattcgcgctatgctgactttccggcagcgttgattcgtgattacaccgatctggaaattctggcagagacggaagaaggggatgcatatctgtttgccagtaaagataagcgcattgcctttgtgacgggccatcccgaatatgatgcgcaaacgctggcgcaggaatttttccgcgatgtggaagccggactagacccggatgtaccgtataactatttcccgcacaatgatccgcaaaatacaccgcgagcgagctggcgtagtcacggtaatttactgtttaccaactggctcaactattacgtctaccagatcacgccatacgatctacggcacatgaatccaacgctggattaa

SEQ ID NO: 54 - protein, 309 aa, Escherichia coli [sequence omitted]

SEQ ID NO: 55 - DNA, 1026 bp, Escherichia coli
  source 1..1026: mol_type = genomic DNA
atgaaagcgttatccaaactgaaagcggaagagggcatctggatgaccgacgttcctgtaccggaactcgggcataacgatctgctgattaaaatccgtaaaacagccatctgcgggactgacgttcacatctataactgggatgagtggtcgcaaaaaaccatcccggtgccgatggtcgtgggccatgaatatgtcggtgaagtggtaggtattggtcaggaagtgaaaggcttcaagatcggcgatcgcgtttctggcgaaggccatatcacctgtggtcattgccgcaactgtcgtggtggtcgtacccatttgtgccgcaacacgataggcgttggtgttaatcgcccgggctgctttgccgaatatctggtgatcccggcattcaacgccttcaaaatccccgacaatatttccgatgacttagccgcaatttttgatcccttcggtaacgccgtgcataccgcgctgtcgtttgatctggtgggcgaagatgtgctggtttctggtgcaggcccgattggtattatggcagcggcggtggcgaaacacgttggtgcacgcaatgtggtgatcactgatgttaacgaataccgccttgagctggcgcgtaaaatgggtatcacccgtgcggttaacgtcgccaaagaaaatctcaatgacgtgatggcggagttaggcatgaccgaaggttttgatgtcggtctggaaatgtccggtgcgccgccagcgtttcgtaccatgcttgacaccatgaatcacggcggccgtattgcgatgctgggtattccgccgtctgatatgtctatcgactggaccaaagtgatctttaaaggcttgttcattaaaggtatttacggtcgtgagatgtttgaaacctggtacaagatggcggcgctgattcagtctggcctcgatctttcgccgatcattacccatcgtttctctatcgatgatttccagaagggctttgacgctatgcgttcgggccagtccgggaaagttattctgagctgggattaa

SEQ ID NO: 56 - protein, 341 aa, Escherichia coli
MKALSKLKAEEGIWMTDVPVPELGHNDLLIKIRKTAICGTDVHIYNWDEWSQKTIPVPMVVGHEYVGEVVGIGQEVKGFKIGDRVSGEGHITCGHCRNCRGGRTHLCRNTIGVGVNRPGCFAEYLVIPAFNAFKIPDNISDDLAAIFDPFGNAVHTALSFDLVGEDVLVSGAGPIGIMAAAVAKHVGARNVVITDVNEYRLELARKMGITRAVNVAKENLNDVMAELGMTEGFDVGLEMSGAPPAFRTMLDTMNHGGRIAMLGIPPSDMSIDWTKVIFKGLFIKGIYGREMFETWYKMAALIQSGLDLSPIITHRFSIDDFQKGFDAMRSGQSGKVILSWD

SEQ ID NO: 57 - DNA, 825 bp, Escherichia coli
  source 1..825: mol_type = genomic DNA
atggtcgcacccattcccgcgaaacgcggcagaaaacccgccgttgccaccgcaccagcgactggacaggttcagtctttaacgcgtggcctgaaattactggagtggattgccgaatccaatggcagtgtggcactcacggaactggcgcaacaagccgggttacccaattccacgacccaccgcctgctaaccacgatgcaacagcagggtttcgtgcgtcaggttggcgaactgggacattgggcaatcggcgcacatgcctttatggtcggcagcagctttctccagagccgtaatttgttagcgattgttcaccctatcctgcgcaatctaatggaagagtctggcgaaacggtcaatatggcggtgcttgatcaaagcgatcacgaagcgattattatcgaccaggtacagtgtacgcatctgatgcgaatgtccgcgcctatcggcggtaaattgccgatgcacgcttccggtgcgggtaaagcctttttagcccaactgagcgaagaacaggtgacgaagctgctgcaccgcaaagggttacatgcctatacccacgcaacgctggtgtctcctgtgcatttaaaagaagatctcgcccaaacgcgcaaacggggttattcatttgacgatgaggaacatgcactggggctacgttgccttgcagcgtgtattttcgatgagcaccgtgaaccgtttgccgcaatttctatttccggaccgatttcacgtattaccgatgaccgcgtgaccgagtttggcgcgatggtgattaaagcggcgaaggaagtgacgctggcgtacggtggaatgcgctga

SEQ ID NO: 58 - protein, 274 aa, Escherichia coli
MVAPIPAKRGRKPAVATAPATGQVQSLTRGLKLLEWIAESNGSVALTELAQQAGLPNSTTHRLLTTMQQQGFVRQVGELGHWAIGAHAFMVGSSFLQSRNLLAIVHPILRNLMEESGETVNMAVLDQSDHEAIIIDQVQCTHLMRMSAPIGGKLPMHASGAGKAFLAQLSEEQVTKLLHRKGLHAYTHATLVSPVHLKEDLAQTRKRGYSFDDEEHALGLRCLAACIFDEHREPFAAISISGPISRITDDRVTEFGAMVIKAAKEVTLAYGGMR

SEQ ID NO: 59 - DNA, 2463 bp, Escherichia coli
  source 1..2463: mol_type = genomic DNA
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctga

SEQ ID NO: 60 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 61 - DNA, 2463 bp, synthetic construct
  misc_feature 1..2463: note = thrA* gene
  source 1..2463: mol_type = other DNA
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctga

SEQ ID NO: 62 - protein, 820 aa, synthetic construct
  REGION 1..820: note = ThrA* protein
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARIFVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 63 - DNA, 1350 bp, Escherichia coli
  source 1..1350: mol_type = genomic DNA
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaacccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 64 - protein, 449 aa, Escherichia coli
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALTLDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 65 - DNA, 1350 bp, synthetic construct
  misc_feature 1..1350: note = lysC* gene
  source 1..1350: mol_type = other DNA
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 66 - protein, 449 aa, synthetic construct
  REGION 1..449: note = LysC* protein
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALILDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 67 - DNA, 1545 bp, Escherichia coli
  source 1..1545: mol_type = genomic DNA
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 68 - protein, 514 aa, Escherichia coli [sequence omitted]

SEQ ID NO: 69 - DNA, 1545 bp, synthetic construct
  misc_feature 1..1545: note = ilvA* gene
  source 1..1545: mol_type = other DNA
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgtttctgcgcttcgccaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 70 - protein, 514 aa, synthetic construct
  REGION 1..514: note = IlvA* protein
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDAVRGFGGEVLLHGANFDEAKAKAIELSQQQGFTWVPPFDHPMVIAGQGTLALELLQQDAHLDRVFVPVGGGGLAAGVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELGEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGAFLRFANTLGTYWNISLFHYRSHGTDYGRVLAAFELGDHEPDFETRLNELGYDCHDETNNPAFRFFLAG

SEQ ID NO: 71 - DNA, 1476 bp, Escherichia coli
  source 1..1476: mol_type = genomic DNA
atggcgaactatttcaatacactgaacttacgccaacagctggcacagcttggaaaatgccgctttatgggacgtgatgaatttgcagacggggcaagttatttacagggcaagaaagttgtgatcgtgggatgcggcgcgcagggtcttaaccagggcctgaatatgcgcgacagcggcctcgacatttcatatgccctgcgcaaggaagcgattgcggaaaagcgtgctagctggcgcaaagcgacagaaaacggttttaaagtaggcacgtacgaagagttaattccgcaagccgatctggtgatcaatctgacgcccgataaacagcacagtgacgttgtgcgcacggtacaaccgcttatgaaagatggcgccgcgttgggttatagccacggcttcaatattgtggaagtgggcgaacagattcgcaaggatattaccgtggttatggtggccccgaaatgccctggaaccgaagtacgtgaagaatataaacgcggttttggcgtaccgacactgattgcggtgcatccggagaatgatccgaagggcgaaggcatggcgattgccaaggcttgggcggcagctacgggcggccatcgcgcaggtgtcctcgaatcttcttttgtggcagaggtcaaatcggatctgatgggcgagcagaccattctgtgtggcatgctgcaagccggctcactgctgtgttttgacaaactggtagaggaaggaaccgatcctgcgtacgctgaaaagctgattcagttcggctgggaaaccattacggaagcccttaaacagggcggaattaccctgatgatggaccgcctgagcaacccggcgaaattgcgcgcctatgcattaagcgaacagttgaaagaaattatggccccgctgttccagaaacatatggatgatattatttctggcgagttctcgtcgggtatgatggccgattgggcaaacgatgacaagaaattgttaacctggcgcgaagaaacgggcaagactgctttcgaaactgcgccgcagtacgaaggcaagattggagaacaggaatacttcgataaaggcgttctgatgatcgcgatggtcaaagcgggcgttgaactggcatttgaaacgatggtggatagcggaatcattgaagaaagcgcctactacgagagcttgcatgaactgccgctgattgccaacaccatcgcgcgtaaacgcctgtatgaaatgaacgtggtgatcagtgacacagcggaatacggtaattatcttttctcatatgcttgtgtgcctcttttgaagccgttcatggccgaactgcaaccaggtgacctgggcaaagccattcccgaaggcgcagtggacaacggccagttacgcgatgtgaacgaggccattcgtagccatgccatcgaacaggtgggaaagaaacttcgtggctacatgaccgatatgaagcgcatcgccgtggccggctaa

SEQ ID NO: 72 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 73 - DNA, 2652 bp, Escherichia coli
  source 1..2652: mol_type = genomic DNA
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 74 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 75 - DNA, 495 bp, Escherichia coli
  source 1..495: mol_type = genomic DNA
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatccggtgatttcgactacctgttgaaaacacgcgtgccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 76 - protein, 164 aa, Escherichia coli
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHLVSGDFDYLLKTRVPDMSAYRKLLGETLLRLPGVNDTRTYVVMEEVKQSNRLVIKTR

SEQ ID NO: 77 - DNA, 1851 bp, Escherichia coli
  source 1..1851: mol_type = genomic DNA
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 78 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 79 - DNA, 1689 bp, Escherichia coli
  source 1..1689: mol_type = genomic DNA
atggcaagttcgggcacaacatcgacgcgtaagcgctttaccggcgcagaatttatcgttcatttcctggaacagcagggcattaagattgtgacaggcattccgggcggttctatcctgcctgtttacgatgccttaagccaaagcacgcaaatccgccatattctggcccgtcatgaacagggcgcgggctttatcgctcagggaatggcgcgcaccgacggtaaaccggcggtctgtatggcctgtagcggaccgggtgcgactaacctggtgaccgccattgccgatgcgcggctggactccatcccgctgatttgcatcactggtcaggttcccgcctcgatgatcggcaccgacgccttccaggaagtggacacctacggcatctctatccccatcaccaaacacaactatctggtcagacatatcgaagaactcccgcaggtcatgagcgatgccttccgcattgcgcaatcaggccgcccaggcccggtgtggatagacattcctaaggatgtgcaaacggcagtttttgagattgaaacacagcccgctatggcagaaaaagccgccgcccccgcctttagcgaagaaagcattcgtgacgcagcggcgatgattaacgctgccaaacgcccggtgctttatctgggcggcggtgtgatcaatgcgcccgcacgggtgcgtgaactggcggagaaagcgcaactgcctaccaccatgactttaatggcgctgggcatgttgccaaaagcgcatccgttgtcgctgggtatgctggggatgcacggcgtgcgcagcaccaactatattttgcaggaggcggatttgttgatagtgctcggtgcgcgttttgatgaccgggcgattggcaaaaccgagcagttctgtccgaatgccaaaatcattcatgtcgatatcgaccgtgcagagctgggtaaaatcaagcagccgcacgtggcgattcaggcggatgttgatgacgtgctggcgcagttgatcccgctggtggaagcgcaaccgcgtgcagagtggcaccagttggtagcggatttgcagcgtgagtttccgtgtccaatcccgaaagcgtgcgatccgttaagccattacggcctgatcaacgccgttgccgcctgtgtcgatgacaatgcaattatcaccaccgacgttggtcagcatcagatgtggaccgcgcaagcttatccgctcaatcgcccacgccagtggctgacctccggtgggctgggcacgatgggttttggcctgcctgcggcgattggcgctgcgctggcgaacccggatcgcaaagtgttgtgtttctccggcgacggcagcctgatgatgaatattcaggagatggcgaccgccagtgaaaatcagctggatgtcaaaatcattctgatgaacaacgaagcgctggggctggtgcatcagcaacagagtctgttctacgagcaaggcgtttttgccgccacctatccgggcaaaatcaactttatgcagattgccgccggattcggcctcgaaacctgtgatttgaataacgaagccgatccgcaggcttcattgcaggaaatcatcaatcgccctggcccggcgctgatccatgtgcgcattgatgccgaagaaaaagtttacccgatggtgccgccaggtgcggcgaatactgaaatggtgggggaataa

SEQ ID NO: 80 - protein, 562 aa, Escherichia coli
MASSGTTSTRKRFTGAEFIVHFLEQQGIKIVTGIPGGSILPVYDALSQSTQIRHILARHEQGAGFIAQGMARTDGKPAVCMACSGPGATNLVTAIADARLDSIPLICITGQVPASMIGTDAFQEVDTYGISIPITKHNYLVRHIEELPQVMSDAFRIAQSGRPGPVWIDIPKDVQTAVFEIETQPAMAEKAAAPAFSEESIRDAAAMINAAKRPVLYLGGGVINAPARVRELAEKAQLPTTMTLMALGMLPKAHPLSLGMLGMHGVRSTNYILQEADLLIVLGARFDDRAIGKTEQFCPNAKIIHVDIDRAELGKIKQPHVAIQADVDDVLAQLIPLVEAQPRAEWHQLVADLQREFPCPIPKACDPLSHYGLINAVAACVDDNAIITTDVGQHQMWTAQAYPLNRPRQWLTSGGLGTMGFGLPAAIGAALANPDRKVLCFSGDGSLMMNIQEMATASENQLDVKIILMNNEALGLVHQQQSLFYEQGVFAATYPGKINFMQIAAGFGLETCDLNNEADPQASLQEIINRPGPALIHVRIDAEEKVYPMVPPGAANTEMVGE

SEQ ID NO: 81 - DNA, 1092 bp, Escherichia coli
  source 1..1092: mol_type = genomic DNA
atgtcgaagaattaccatattgccgtattgccgggggacggtattggtccggaagtgatgacccaggcgctgaaagtgctggatgccgtgcgcaaccgctttgcgatgcgcatcaccaccagccattacgatgtaggcggcgcagccattgataaccacgggcaaccactgccgcctgcgacggttgaaggttgtgagcaagccgatgccgtgctgtttggctcggtaggcggcccgaagtgggaacatttaccaccagaccagcaaccagaacgcggcgcgctgctgcctctgcgtaagcacttcaaattattcagcaacctgcgcccggcaaaactgtatcaggggctggaagcattctgtccgctgcgtgcagacattgccgcaaacggcttcgacatcctgtgtgtgcgcgaactgaccggcggcatctatttcggtcagccaaaaggccgcgaaggtagcggacaatatgaaaaagcctttgataccgaggtgtatcaccgttttgagatcgaacgtatcgcccgcatcgcgtttgaatctgctcgcaagcgtcgccacaaagtgacgtcgatcgataaagccaacgtgctgcaatcctctattttatggcgggagatcgttaacgagatcgccacggaatacccggatgtcgaactggcgcatatgtacatcgacaacgccaccatgcagctgattaaagatccatcacagtttgacgttctgctgtgctccaacctgtttggcgacattctgtctgacgagtgcgcaatgatcactggctcgatggggatgttgccttccgccagcctgaacgagcaaggttttggactgtatgaaccggcgggcggctcggcaccagatatcgcaggcaaaaacatcgccaacccgattgcacaaatcctttcgctggcactgctgctgcgttacagcctggatgccgatgatgcggcttgcgccattgaacgcgccattaaccgcgcattagaagaaggcattcgcaccggggatttagcccgtggcgctgccgccgttagtaccgatgaaatgggcgatatcattgcccgctatgtagcagaaggggtgtaa

SEQ ID NO: 82 - protein, 363 aa, Escherichia coli
MSKNYHIAVLPGDGIGPEVMTQALKVLDAVRNRFAMRITTSHYDVGGAAIDNHGQPLPPATVEGCEQADAVLFGSVGGPKWEHLPPDQQPERGALLPLRKHFKLFSNLRPAKLYQGLEAFCPLRADIAANGFDILCVRELTGGIYFGQPKGREGSGQYEKAFDTEVYHRFEIERIARIAFESARKRRHKVTSIDKANVLQSSILWREIVNEIATEYPDVELAHMYIDNATMQLIKDPSQFDVLLCSNLFGDILSDECAMITGSMGMLPSASLNEQGFGLYEPAGGSAPDIAGKNIANPIAQILSLALLLRYSLDADDAACAIERAINRALEEGIRTGDLARGAAAVSTDEMGDIIARYVAEGV

SEQ ID NO: 83 - DNA, 1401 bp, Escherichia coli
  source 1..1401: mol_type = genomic DNA
atggctaagacgttatacgaaaaattgttcgacgctcacgttgtgtacgaagccgaaaacgaaaccccactgttatatatcgaccgccacctggtgcatgaagtgacctcaccgcaggcgttcgatggtctgcgcgcccacggtcgcccggtacgtcagccgggcaaaaccttcgctaccatggatcacaacgtctctacccagaccaaagacattaatgcctgcggtgaaatggcgcgtatccagatgcaggaactgatcaaaaactgcaaagaatttggcgtcgaactgtatgacctgaatcacccgtatcaggggatcgtccacgtaatggggccggaacagggcgtcaccttgccggggatgaccattgtctgcggcgactcgcataccgccacccacggcgcgtttggcgcactggcctttggtatcggcacttccgaagttgaacacgtactggcaacgcaaaccctgaaacagggccgcgcaaaaaccatgaaaattgaagtccagggcaaagccgcgccgggcattaccgcaaaagatatcgtgctggcaattatcggtaaaaccggtagcgcaggcggcaccgggcatgtggtggagttttgcggcgaagcaatccgtgatttaagcatggaaggtcgtatgaccctgtgcaatatggcaatcgaaatgggcgcaaaagccggtctggttgcaccggacgaaaccacctttaactatgtcaaaggccgtctgcatgcgccgaaaggcaaagatttcgacgacgccgttgcctactggaaaaccctgcaaaccgacgaaggcgcaactttcgataccgttgtcactctgcaagcagaagaaatttcaccgcaggtcacctggggcaccaatcccggccaggtgatttccgtgaacgacaatattcccgatccggcttcgtttgccgatccggttgaacgcgcgtcggcagaaaaagcgctggcctatatggggctgaaaccgggtattccgctgaccgaagtggctatcgacaaagtgtttatcggttcctgtaccaactcgcgcattgaagatttacgcgcggcagcggagatcgccaaagggcgaaaagtcgcgccaggcgtgcaggcactggtggttcccggctctggcccggtaaaagcccaggcggaagcggaaggtctggataaaatctttattgaagccggttttgaatggcgcttgcctggctgctcaatgtgtctggcgatgaacaacgaccgtctgaatccgggcgaacgttgtgcctccaccagcaaccgtaactttgaaggccgccaggggcgcggcgggcgcacgcatctggtcagcccggcaatggctgccgctgctgctgtgaccggacatttcgccgacattcgcaacattaaataa

SEQ ID NO: 84 - protein, 466 aa, Escherichia coli
MAKTLYEKLFDAHVVYEAENETPLLYIDRHLVHEVTSPQAFDGLRAHGRPVRQPGKTFATMDHNVSTQTKDINACGEMARIQMQELIKNCKEFGVELYDLNHPYQGIVHVMGPEQGVTLPGMTIVCGDSHTATHGAFGALAFGIGTSEVEHVLATQTLKQGRAKTMKIEVQGKAAPGITAKDIVLAIIGKTGSAGGTGHVVEFCGEAIRDLSMEGRMTLCNMAIEMGAKAGLVAPDETTFNYVKGRLHAPKGKDFDDAVAYWKTLQTDEGATFDTVVTLQAEEISPQVTWGTNPGQVISVNDNIPDPASFADPVERASAEKALAYMGLKPGIPLTEVAIDKVFIGSCTNSRIEDLRAAAEIAKGRKVAPGVQALVVPGSGPVKAQAEAEGLDKIFIEAGFEWRLPGCSMCLAMNNDRLNPGERCASTSNRNFEGRQGRGGRTHLVSPAMAAAAAVTGHFADIRNIK

SEQ ID NO: 85 - DNA, 606 bp, Escherichia coli
  source 1..606: mol_type = genomic DNA
atggcagagaaatttatcaaacacacaggcctggtggttccgctggatgccgccaatgtcgataccgatgcaatcatcccgaaacagtttttgcagaaagtgacccgtacgggttttggcgcgcatctgtttaacgactggcgttttctggatgaaaaaggccaacagccaaacccggacttcgtgctgaacttcccgcagtatcagggcgcttccattttgctggcacgagaaaacttcggctgtggctcttcgcgtgagcacgcgccctgggcattgaccgactacggttttaaagtggtgattgcgccgagttttgctgacatcttctacggcaatagctttaacaaccagctgctgccggtgaaattaagcgatgcagaagtggacgaactgtttgcgctggtgaaagctaatccggggatccatttcgacgtggatctggaagcgcaagaggtgaaagcgggagagaaaacctatcgctttaccatcgatgccttccgccgccactgcatgatgaacggtctggacagtattgggcttaccttgcagcacgacgacgccattgccgcttatgaagcaaaacaacctgcgtttatgaattaa

SEQ ID NO: 86 - protein, 201 aa, Escherichia coli
MAEKFIKHTGLVVPLDAANVDTDAIIPKQFLQKVTRTGFGAHLFNDWRFLDEKGQQPNPDFVLNFPQYQGASILLARENFGCGSSREHAPWALTDYGFKVVIAPSFADIFYGNSFNNQLLPVKLSDAEVDELFALVKANPGIHFDVDLEAQEVKAGEKTYRFTIDAFRRHCMMNGLDSIGLTLQHDDAIAAYEAKQPAFMN

SEQ ID NO: 87 - DNA, 492 bp, Escherichia coli
  source 1..492: mol_type = genomic DNA
atgcgccggatattatcagtcttactcgaaaatgaatcaggcgcgttatcccgcgtgattggccttttttcccagcgtggctacaacattgaaagcctgaccgttgcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttga

SEQ ID NO: 88 - protein, 163 aa, Escherichia coli
MRRILSVLLENESGALSRVIGLFSQRGYNIESLTVAPTDDPTLSRMTIQTVGDEKVLEQIEKQLHKLVDVLRVSELGQGAHVEREIMLVKIQASGYGRDEVKRNTEIFRGQIIDVTPSLYTVQLAGTSGKLDAFLASIRDVAKIVEVARSGVVGLSRGDKIMR

SEQ ID NO: 89 - DNA, 492 bp, synthetic construct
  misc_feature 1..492: note = ilvH* gene
  source 1..492: mol_type = other DNA
atgcgccggatattatcagtcttactcgaaaatgaatcagacgcgttattccgcgtgattggccttttttcccagcgtggctacaacattgaaagcctgaccgttgcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttga

SEQ ID NO: 90 - protein, 163 aa, synthetic construct
  REGION 1..163: note = IlvH* protein
MRRILSVLLENESDALFRVIGLFSQRGYNIESLTVAPTDDPTLSRMTIQTVGDEKVLEQIEKQLHKLVDVLRVSELGQGAHVEREIMLVKIQASGYGRDEVKRNTEIFRGQIIDVTPSLYTVQLAGTSGKLDAFLASIRDVAKIVEVARSGVVGLSRGDKIMR

SEQ ID NO: 91 - DNA, 933 bp, Escherichia coli
  source 1..933: mol_type = genomic DNA
atggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaa

SEQ ID NO: 92 - protein, 310 aa, Escherichia coli
MVKVYAPASSANMSVGFDVLGAAVTPVDGALLGDVVTVEAAETFSLNNLGRFADKLPSEPRENIVYQCWERFCQELGKQIPVAMTLEKNMPIGSGLGSSACSVVAALMAMNEHCGKPLNDTRLLALMGELEGRISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 93 - DNA, 1287 bp, Escherichia coli
  source 1..1287: mol_type = genomic DNA
atgaaactctacaatctgaaagatcacaacgagcaggtcagctttgcgcaagccgtaacccaggggttgggcaaaaatcaggggctgttttttccgcacgacctgccggaattcagcctgactgaaattgatgagatgctgaagctggattttgtcacccgcagtgcgaagatcctctcggcgtttattggtgatgaaatcccacaggaaatcctggaagagcgcgtgcgcgcggcgtttgccttcccggctccggtcgccaatgttgaaagcgatgtcggttgtctggaattgttccacgggccaacgctggcatttaaagatttcggcggtcgctttatggcacaaatgctgacccatattgcgggtgataagccagtgaccattctgaccgcgacctccggtgataccggagcggcagtggctcatgctttctacggtttaccgaatgtgaaagtggttatcctctatccacgaggcaaaatcagtccactgcaagaaaaactgttctgtacattgggcggcaatatcgaaactgttgccatcgacggcgatttcgatgcctgtcaggcgctggtgaagcaggcgtttgatgatgaagaactgaaagtggcgctagggttaaactcggctaactcgattaacatcagccgtttgctggcgcagatttgctactactttgaagctgttgcgcagctgccgcaggagacgcgcaaccagctggttgtctcggtgccaagcggaaacttcggcgatttgacggcgggtctgctggcgaagtcactcggtctgccggtgaaacgttttattgctgcgaccaacgtgaacgataccgtgccacgtttcctgcacgacggtcagtggtcacccaaagcgactcaggcgacgttatccaacgcgatggacgtgagtcagccgaacaactggccgcgtgtggaagagttgttccgccgcaaaatctggcaactgaaagagctgggttatgcagccgtggatgatgaaaccacgcaacagacaatgcgtgagttaaaagaactgggctacacttcggagccgcacgctgccgtagcttatcgtgcgctgcgtgatcagttgaatccaggcgaatatggcttgttcctcggcaccgcgcatccggcgaaatttaaagagagcgtggaagcgattctcggtgaaacgttggatctgccaaaagagctggcagaacgtgctgatttacccttgctttcacataatctgcccgccgattttgctgcgttgcgtaaattgatgatgaatcatcagtaa

SEQ ID NO: 94 - protein, 428 aa, Escherichia coli
MKLYNLKDHNEQVSFAQAVTQGLGKNQGLFFPHDLPEFSLTEIDEMLKLDFVTRSAKILSAFIGDEIPQEILEERVRAAFAFPAPVANVESDVGCLELFHGPTLAFKDFGGRFMAQMLTHIAGDKPVTILTATSGDTGAAVAHAFYGLPNVKVVILYPRGKISPLQEKLFCTLGGNIETVAIDGDFDACQALVKQAFDDEELKVALGLNSANSINISRLLAQICYYFEAVAQLPQETRNQLVVSVPSGNFGDLTAGLLAKSLGLPVKRFIAATNVNDTVPRFLHDGQWSPKATQATLSNAMDVSQPNNWPRVEELFRRKIWQLKELGYAAVDDETTQQTMRELKELGYTSEPHAAVAYRALRDQLNPGEYGLFLGTAHPAKFKESVEAILGETLDLPKELAERADLPLLSHNLPADFAALRKLMMNHQ

SEQ ID NO: 95 - DNA, 738 bp, Escherichia coli
  source 1..738: mol_type = genomic DNA
atggaaagccctactccacagcctgctcctggttcggcgaccttcatggaaggatgcaaagacagtttaccgattgttattagttatattccggtggcctttgcgttcggtctgaatgcgacccgtctgggattctctcctctcgaaagcgtttttttctcctgcatcatttatgcaggcgcgagccagttcgtcattaccgcgatgctggcagccgggagtagtttgtggattgctgcactgaccgtcatggcaatggatgttcgccatgtgttgtatggcccgtcactgcgtagccgtattattcagcgtctgcaaaaatcgaaaaccgccctgtgggcgtttggcctgacggatgaggtttttgccgccgcaaccgcaaaactggtacgcaataatcgccgctggagcgagaactggatgatcggcattgccttcagttcatggtcatcgtgggtatttggtacggtaataggggcattctccggcagcggcttgctgcaaggttatcccgccgttgaagctgcattaggttttatgcttccggcactctttatgagtttcctgctcgcctctttccagcgcaaacaatctctttgcgttaccgcagcgttagttggtgcccttgcaggcgtaacgctattttctattcccgtcgccattctggcaggcattgtctgtggctgcctcactgcgttaatccaggcattctggcaaggagcgcccgatgagctatga

SEQ ID NO: 96 - protein, 245 aa, Escherichia coli
MESPTPQPAPGSATFMEGCKDSLPIVISYIPVAFAFGLNATRLGFSPLESVFFSCIIYAGASQFVITAMLAAGSSLWIAALTVMAMDVRHVLYGPSLRSRIIQRLQKSKTALWAFGLTDEVFAAATAKLVRNNRRWSENWMIGIAFSSWSSWVFGTVIGAFSGSGLLQGYPAVEAALGFMLPALFMSFLLASFQRKQSLCVTAALVGALAGVTLFSIPVAILAGIVCGCLTALIQAFWQGAPDEL

SEQ ID NO: 97 - DNA, 336 bp, Escherichia coli
  source 1..336: mol_type = genomic DNA
atgagctatgaggttctgctgcttgggttactagttggcgtggcgaattattgcttccgctatttgccgctgcgcctgcgtgtgggtaatgcccgcccaaccaaacgtggcgcggtaggtattttgctcgacaccattggcatcgcctcgatatgcgctctgctggttgtctctaccgcaccagaagtgatgcacgatacacgccgtttcgtgcccacgctggtcggcttcgcggtactgggtgccagtttctataaaacacgcagcattatcatcccaacactgcttagtgcgctggcctatgggctcgcctggaaagtgatggcgattatataa

SEQ ID NO: 98 - protein, 111 aa, Escherichia coli
MSYEVLLLGLLVGVANYCFRYLPLRLRVGNARPTKRGAVGILLDTIGIASICALLVVSTAPEVMHDTRRFVPTLVGFAVLGASFYKTRSIIIPTLLSALAYGLAWKVMAII

SEQ ID NO: 99 - DNA, 1809 bp, Escherichia coli
  source 1..1809: mol_type = genomic DNA
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 100 - protein, 602 aa, Escherichia coli
VQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 101 - DNA, 735 bp, Escherichia coli
  source 1..735: mol_type = genomic DNA
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 102 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 103 - DNA, 396 bp, Escherichia coli
  source 1..396: mol_type = genomic DNA
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 104 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 105 - DNA, 360 bp, Escherichia coli
  source 1..360: mol_type = genomic DNA
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

SEQ ID NO: 106 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 107 - DNA, 741 bp, Escherichia coli
  source 1..741: mol_type = genomic DNA
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 108 - protein, 246 aa, Escherichia coli
MSVIGRIHSFESCGTVDGPGIRFITFFQGCLMRCLYCHNRDTWDTHGGKEVTVEDLMKEVVTYRHFMNASGGGVTASGGEAILQAEFVRDWFRACKKEGIHTCLDTNGFVRRYDPVIDELLEVTDLVMLDLKQMNDEIHQNLVGVSNHRTLEFAKYLANKNVKVWIRYVVVPGWSDDDDSAHRLGEFTRDMGNVEKIELLPYHELGKHKWVAMGEEYKLDGVKPPKKETMERVKGILEQYGHKVMF

SEQ ID NO: 109 - DNA, 2283 bp, Escherichia coli
  source 1..2283: mol_type = genomic DNA
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 110 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 111 - DNA, 1203 bp, Escherichia coli
  source 1..1203: mol_type = genomic DNA
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccttacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctgaccaaagagtctggcctgctgggtctgaccgaagtgaccagcgactgccgctatgttgaagacaactacgcgacgaaagaagacgcgaagcgcgcaatggacgtttactgccaccgcctggcgaaatacatcggtgcctacactgcgctgatggatggtcgtctggacgctgttgtattcactggtggtatcggtgaaaatgccgcaatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 112 - protein, 400 aa, Escherichia coli
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA

SEQ ID NO: 113 - DNA, 2145 bp, Escherichia coli
  source 1..2145: mol_type = genomic DNA
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 114 - protein, 714 aa, Escherichia coli
VSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 115 - DNA, 639 bp, Escherichia coli
  source 1..639: mol_type = genomic DNA
gtgttcgctgaatacggggttctgaattactggacctatctggttggggccatttttattgtgttggtgccagggccaaataccctgtttgtactcaaaaatagcgtcagtagcggtatgaaaggcggttatcttgcggcctgcggtgtatttattggcgatgcggtattgatgtttctggcatgggctggagtggcgacattaattaagaccaccccgatattattcaacattgtacgttatcttggtgcgttttatttgctctatctggggagtaaaattctttacgcgaccctgaagggtaaaaatagcgaggccaaatccgatgagccccaatacggtgctatttttaaacgcgcgttaattttgagcctgactaatccgaaagccattttgttctatgtgtcgtttttcgtacagtttatcgatgttaatgccccacatacgggaatttcattctttattctggcggcgacgctggaactggtgagtttctgctatttgagcttcctgattatatctggtgcttttgtcacgcagtacatacgtaccaaaaagaaactggctaaagttggcaactcactgattggtttgatgttcgtgggtttcgctgcccgactggcgacgctgcaatcctga

SEQ ID NO: 116 - protein, 212 aa, Escherichia coli
MFAEYGVLNYWTYLVGAIFIVLVPGPNTLFVLKNSVSSGMKGGYLAACGVFIGDAVLMFLAWAGVATLIKTTPILFNIVRYLGAFYLLYLGSKILYATLKGKNSEAKSDEPQYGAIFKRALILSLTNPKAILFYVSFFVQFIDVNAPHTGISFFILAATLELVSFCYLSFLIISGAFVTQYIRTKKKLAKVGNSLIGLMFVGFAARLATLQS